SEQUENCE LISTING (8 sequences)

SEQ ID NO: 1 - protein, 299 aa, Homo sapiens
MQIPQAPWPVVWAVLQLGWRPGWFAEQKLISEEDLLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 10 aa, Homo sapiens
EQKLISEEDL

SEQ ID NO: 3 - protein, 278 aa, Artificial Sequence
  scFv protein sequence (X indicates CDRH3 and CDRL3 sequences)
  misc_feature (117)..(124): Xaa can be any naturally occurring amino acid
  misc_feature (237)..(249): Xaa can be any naturally occurring amino acid
MWMKIKTGARILALSALTTMMFSASALADIQMTQSPSSLSASVGDRVTITCRASQSVSSAVAWYQQKPGKAPKLLIYSASSLYSCVPSRFSGSRSGTDFTLTISSLQPEDFATYYCXXXXXXXXFGQGTKVGGSSRSSEVQLVESGGGLVQPGGSLRLSCAASGFNLYYSYIHWVRQAPGKGLEWVASISPSYGYTSYADSVKGRFTISADTSKNTAYLQMNSLRAEDTAVYYCARXXXXXXXXXXXXXGFDYWGQGTLVTVSSTSLEYYPYDVPDYA

SEQ ID NO: 4 - protein, 17 aa, Artificial Sequence
  Amino acid linker
GGSSRSSEVQLVESGGG

SEQ ID NO: 5 - protein, 297 aa, Artificial Sequence
  Pembrolizumab
MGWSCIILFLVATATGVHSELVMTQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRLEYYPYDVPDYA

SEQ ID NO: 6 - protein, 15 aa, Artificial Sequence
  Amino acid linker
GGGGSGGGGSGGGGS

SEQ ID NO: 7 - DNA, 26 bp, Artificial Sequence
  PCR primer
cgatatcaaaggaggtacccaacatg

SEQ ID NO: 8 - DNA, 31 bp, Artificial Sequence
  PCR primer
cgcataatccggcacatcatacggatagtac

(19) United States
(12) Patent Application Publication
(10) Pub. No.: US 2022/0154174 A1
(43) Pub. Date: May 19, 2022

(54) Method of Selecting for Antibodies

(71) Applicant: Oxford Genetics Limited, Oxford, Oxfordshire (GB)

(72) Inventors: Nancy Lopez-Anton, Oxford, Oxfordshire (GB); Nathan Robertson, Oxford, Oxfordshire (GB); Timothy Jones, Oxford, Oxfordshire (GB); Ryan Cawood, Oxford, Oxfordshire (GB); Thomas Payne, Oxford, Oxfordshire (GB); Richard Parker-Manuel, Oxford, Oxfordshire (GB)

(21) Appl. No.: 17/435,851

(22) PCT Filed: Mar. 6, 2020
(86) PCT No.: PCT/GB2020/050538

§ 371 Date: Sep. 2, 2021

(30) Foreign Application Priority Data
Mar. 8, 2019 (GB) 1903233.3
Mar. 11, 2019 (GB) 1903270.5
Sep. 16, 2019 (GB) 1913333.9
Oct. 14, 2019 (GB) 1914819.6

(51) Int. Cl. C12N 15/10; C12N 15/86; G01N 33/542; C07K 16/30; C07K 16/28

(52) U.S. Cl. CPC C12N 15/1065; C12N 15/86; G01N 33/542; C07K 16/30; C07K 16/286; C07K 2317/92; C12N 2740/15043; C12N 2740/10043; C12N 2830/006; C07K 2317/622; C07K 16/2818

(57) ABSTRACT

The present invention relates to a method for identifying specific binding partners (e.g. antibodies or antibody mimetics) which bind to a desired target polypeptide. In particular, the method involves expressing a library of specific binding partners in a population of mammalian cells, wherein each cell in the population of cells displays the target polypeptide on the outer surface of the cell, and identifying or isolating cells within the population of cells to which specific binding partners are bound.

### CROSS-REFERENCE

This application is a 371 U.S. national phase of PCT/GB2020/050538, filed Mar. 6, 2020, which claims priority from GB1903233.3, filed Mar. 8, 2019; GB1903270.5, filed Mar. 11, 2019; GB1913333.9, filed Sep. 16, 2019; and GB1914819.6, filed Oct. 14, 2019, all which are incorporated by reference in their entirety.

### FIELD OF THE INVENTION

The present invention relates to a method for identifying specific binding partners (e.g. antibodies or antibody mimetics) which bind to a desired target polypeptide. In particular, the method involves expressing a library of specific binding partners in a population of mammalian cells, wherein each cell in the population of cells displays the target polypeptide on the outer surface of the cell, and identifying or isolating cells within the population of cells to which specific binding partners are bound.

### BACKGROUND OF THE INVENTION

Since the invention of hybridoma technology in 1986, monoclonal antibodies have emerged as powerful and versatile biological therapeutics, combining target selectivity, potency, good biological and delivery half-lives and relatively simple large scale manufacture. Today, over eighty monoclonal antibodies are licensed for medical use in the US and Europe, and many others are in development. They are used in the treatment of a broad range of diseases, including inflammation (e.g. rheumatoid arthritis, Crohn's disease, ulcerative colitis, etc.), organ transplantation, asthma, cancer and leukaemia, viral and bacterial infections, aberrant blood clotting and many others.

In medical terms, monoclonal antibodies are usually well-tolerated, with few side-effects, and can have life-changing medical benefits. However, the increasing appreciation of their therapeutic potential has created a rising demand for new monoclonal antibodies against a broad range of targets. This in turn has highlighted the difficulties faced in defining monoclonal antibodies with sufficient potency against challenging targets, most notably against molecules on the cell surface such as integrated membrane proteins. These targets need to retain their physiological configuration during antibody selection, and this severely restricts the strategies that can be used to produce monoclonal antibodies that recognise them (Jones, M. et al., Scientific Reports, 6, 26240 (2016)). It is particularly challenging to define naturally-occurring human antibodies that bind to human targets, because of clonal deletion of many self-recognising antibodies during development.

GPCRs have been historically hard to produce monoclonal antibodies against, due to their need to stay membrane-associated in order to retain their configuration. GPCRs constitute the largest family of membrane protein in humans and are responsible for cellular responses to hormones and neurotransmitters, light sensing, olfaction and taste. About half of current low molecular weight drugs target GPCRs; however, few monoclonal antibodies are in development—even for research—because they are elusive to target. One good example is DRD1 (D1 subtype of the dopamine receptor) which regulates neuronal growth and development, some behavioural responses and modulates DRD2 activity. DRD1 deregulation is thought to play a role in schizophrenia, Huntington's Disease, Parkinson's Disease, hypertension, Alzheimer's Disease and many others. The GPCR market is currently estimated at $1.6 bn (http://www.transparencymarketresearch.com/g-protein-coupled-receptors-market.html). Of the 47 drugs approved that target DRD1, none are monoclonal antibodies. This illustrates the challenge of making effective monoclonal antibodies recognising membrane antigens in their native configuration.

Cancer checkpoint inhibitor antibodies are currently the most exciting new aspect of cancer research, with several agents already licensed against a variety of targets. The market for these is predicted to reach a staggering $19 bn in 2022 (http://immunecheckpoint-europe.com/partner/sponsorship-opportunities/). The one feature these targets share is that they are all membrane antigens (e.g. PD1, PDL1, CTLA4, etc.).

Antibody display may be used to screen for antibodies against a particular target polypeptide. Existing technologies for antibody display include phage display, yeast display, mammalian display, ribosome display, cis-activity based (CIS) display and covalent antibody display (CAD). These technologies all have the same limitation in that membrane target polypeptides (‘baits’) are not presented in their native folded membrane-bound state.

The classical method for high-throughput screening for protein interactions is phage display. In this system, an antibody library is fused to the gene for a bacteriophage coat protein. The library is then transformed into an E. coli host strain (using a phagemid), resulting in a population of phage particles, each containing the sequence for an antibody within its genome and displaying the protein itself on its surface. At first, the phage library is screened with the targeted antigen, immobilised to a surface. Then, unbound phages are washed away; bound phages are recovered and infected into bacteria, and subsequently amplified for library enrichment. This process is normally repeated several times, yielding sequences of gradually improving affinity for the target.

The protein sequence (and level of emerging consensus or homology) of phage in the library can be determined by isolating individual colonies and sequencing their DNA in the appropriate region.

Other systems use a similar concept: for instance, Isogenica's proprietary CIS in vitro display-technology uses the ability of a protein called RepA to bind to its own DNA sequence, allowing it to act as a linker between phenotype and genotype. The advantage of this system is that it facilitates rapid recovery of the antibody encoding DNA sequence after the selection step. However, a significant disadvantage of this system is that the bait protein must be immobilised to a solid support during the in vitro selection step. This precludes the targeting of certain proteins such as large multi-pass membrane proteins.

Cell-surface display is the expression of antibody proteins on the surface of living cells by fusing them to functional components of cells which are exposed to the extracellular milieu. The principle of cell surface display is analogous to phage display, with the recombinant antibody anchored to the cell's surface and the encoding DNA residing within the cell. One advantage of cell-surface display is that cells are large enough to be screened by flow cytometry. In contrast to acellular approaches, the antigen, labelled with a fluorophore, is incubated with the cell-displayed antibody library in solution and then any antigen binding cells are then isolated by fluorescence-activated cell sorting (FACS). Display strategies have been developed for use with bacterial, yeast and mammalian cells. The advantage of using mammalian cells is their ability to express and fold the antibodies in one's library with high fidelity and even with proper post-translational modifications.

One disadvantage of cellular display technologies is that only a relatively small library size is possible compared to acellular technologies due to the limitations of transfecting the library into cells.

The key disadvantage of mammalian cell display, however, is that the antigen has to be in solution. This limits the antigen to relatively small, hydrophilic proteins, essentially excluding large multi-pass membrane proteins, an important class of targets for antibody discovery. Attempts to address this include presenting the antigen in the context of membranous vesicles, but this approach is laborious and so far not very successful.

Chen Zhou's group have developed a mammalian display system for screening full-length antibody cDNA-based libraries (Zhou et al., Acta Biochimica et Biophysica Sinica, 42(8), 575-84.2010; US 2012/0101000). Their system expresses human antibody heavy and light chains together on the surface of mammalian cells; a transmembrane domain from platelet-derived growth factor receptor is fused to the heavy chain to anchor the antibody to the membrane of the cell that expressed it. A human heavy chain (IgG-1) library was constructed separately by RT-PCR amplifying the variable domain from PBMCs and cloning it into a plasmid vector. A human light chain (kappa) library was similarly constructed and the system was used successfully to select antibodies against soluble target antigens. This demonstrates that it is possible to use full-length antibody libraries at the scale necessary to do screening against targets in mammalian cells. However, their approach cannot obtain antibodies against complex membrane bound targets (most commonly required) as it requires a soluble protein for bioselection.

WO 2018/167481 discloses a basic strategy for selection of specific binding partners which recognise target proteins on cells. The approach improved on existing strategies by expressing the target protein on the surface of cells which secrete a library of polypeptide binding partners, e.g. antibodies or antibody mimetics, and then isolating cells that self-label.

One advantage of this approach is that the membrane-bound target polypeptide passes through the proper cellular folding and membrane-insertion pathways before presentation on the surface of the cells. The segment of the membrane-bound target polypeptide presented to the polypeptide binding partners (e.g. antibodies/mimetics) in the library is the same as that which would be available to be bound in an in vivo (cell culture or therapeutic) setting. The ability to select binding partners that bind to membrane proteins is important because membrane proteins in general, including immune checkpoints and G-protein coupled receptors, are key therapeutic targets.

There remains, however, a need to improve on the method of WO2018/167481.

### SUMMARY OF THE INVENTION

The present invention relates to a method for identifying specific binding partners (e.g. antibodies or antibody mimetics) which bind to a desired target polypeptide. In particular, the method involves expressing a library of specific binding partners in a population of mammalian cells, wherein each cell in the population of cells displays the target polypeptide on the outer surface of the cell, and identifying or isolating cells within the population of cells to which specific binding partners are bound.

The method of the invention provides a number of novel features.

Nucleic acid sequences encoding the specific binding partner and the target polypeptide are both integrated into the genome of the host cell.

Integration of the nucleic acid sequence encoding the specific binding partner is achieved using a retroviral vector, preferably a lentiviral vector. Retroviruses can be used to infect cells with high efficiency, thus ensuring that a high proportion of cells will express a binding partner. This is particularly important when considering that the size of the libraries of binding partners may be in excess of 10⁹ members and the consequent necessity to manipulate large numbers of cells. In particular, lentiviruses tend to integrate into regions of active chromatin, thus ensuring that each binding partner will be expressed sufficiently for detection.

Integration of the nucleic acid sequence encoding the target polypeptide is achieved by site-specific recombination. This saves time and provides more uniform expression of the target polypeptide.

A detectable tag is attached to the specific binding partner. This facilitates easier detection of cells to which the specific binding partner is bound.

The cells to which specific binding partners are bound are isolated or detected by flow cytometry (e.g. FACS), by magnetic sorting (e.g. MACS) or by using a micro-fluidics system wherein individual cells from the population of mammalian cells are contained within a plurality of isolated chambers (e.g. droplets or pens).

It is anticipated that members of the binding partner library will be transcribed and translated, and transported to the ER and secretory pathway, where they will encounter the target polypeptide and may bind even before the target polypeptide is transported to the cell surface. However, it is probable that there will be an excess of the binding partner that will be secreted into the culture medium and will therefore have the opportunity to bind to target polypeptide-expressing cells other than the one that is producing the binding partner.

The Applicant has recognised that improvements to the sensitivity of the method may be achieved by reducing the level of cross-binding between binding partners and cells which do not produce those binding partners.

With regard to flow cytometry and magnetic sorting systems in particular (e.g. FACS and MACS), this cross-binding may be reduced by the timing of the selection process, e.g. by assaying for the binding of specific binding partners at a time point when the cells first become bound by internally-produced binding partners. In some embodiments, the removal of cells from the population of cells to which (non-specific) binding partners are bound before induction of expression of the target polypeptide also contributes to reducing the level of non-specific binding.

The Applicants have now found that cross-labelling may be reduced significantly by using a micro-fluidics system. In such systems, one or more cells from the population of cells are contained within a plurality of isolated chambers (such as within droplets or in pens on the surface of a chip), together with the necessary detection reagents, such that each individual cell or small numbers of cells can be assayed independently for the production of a target polypeptide-specific binding partner in isolation from other cells displaying the target polypeptide on their surface.

For example, cells may be encapsulated in the small droplets of stabilised oil/water emulsions, each droplet being of 200 pL or less in volume. Cell-containing droplets can be passed at high speed through microfluidic channels for analysis by lasers for specific fluorescence that indicates cell surface labelling; positive droplets can then be isolated and dispensed into microtitre plates for recovery and expansion of positive individual cells. An example of such an instrument is the Cyto-Mine@ manufactured by Sphere Fluidics (Cambridge, UK).

An alternative micro-fluidics approach is to use optofluidic technology that uses light and millions of light-actuated pixels to move individual cells into picolitre sized reaction chambers (e.g. NanoPens™) arrayed on a chip such that cells can be assayed in isolation in the reaction chambers for desired properties using fluorescence imaging of each pen. An example of such an instrument is the Beacon platform produced by Berkeley Lights (Emeryville, Calif.).

Once cells to which specific binding partners bind have been identified, the nucleotide sequences of the specific binding partners in those cells may be sequenced using non-Sanger based approaches. This allows the sequencing of pools of cells, rather than just individual cells.

### DESCRIPTION OF THE DRAWINGS

FIG. 1: Labelling of cells expressing the EpCAM protein with an anti-EpCAM single chain antibody. The bar graph shows the median fluorescence intensity of each population of cells after staining with a fluorescently-labelled anti HA-antibody and subsequent flow cytometry analysis. HEK293 cells were co-transfected with EpCAM and an anti-EpCAM single chain antibody (top bar). Controls included cells transfected with either EpCAM or the antibody alone or with empty vector (bottom).

FIGS. 2A to 2D: Cells expressing EpCAM and GFP were mixed with cells expressing EpCAM and anti-EpCAM scFv. The X-axis indicates the degree of scFv labelling whereas the Y-axis indicates GFP fluorescence intensity. Cells expressing GFP but not scFv appear in the upper left (UL) quadrant; cells self-labelled with scFv but negative for GFP appear in the lower right (LR) quadrant; GFP positive cells that have become trans-labelled with soluble scFv from the scFv expressing-cells appear in the upper right (UR) quadrant. At higher ratios of EpCAM-GFP to EpCAM-scFv cells (25:1 and 125:1), a small sub-population of self-labelled cells is observed before any detectable antibody has had chance to transfer to other cells in the population.

FIG. 3: One example of the method of the invention, showing antibodies recognising integrated membrane proteins. A. Co-expression of bait protein on human cell surface (open dot) alongside secreted scFv library (average one scFv per cell). B. A cell that expresses a scFV that binds to the bait becomes self-labelled. C. Cells are stained for surface-bound scFv with a fluorescent secondary antibody (starred). D. Fluorescent cells are sorted into individual wells of a microtiter plate by FACS. Supematant binding activity is characterised and lead candidates are sequenced prior to affinity maturation.

FIG. 4: One example of a target polypeptide (bait) expression construct.

FIG. 5: One example of a scFv expression construct. Expression is driven by the spleen focus-forming virus (SFFV) promoter. The expression construct encodes a C-terminal Human influenza haemagglutinin (HA)-tag.

FIG. 6. A lentiviral payload vector consisting of an expression cassette containing PD-1 flanked by lentiviral LTRs.

FIG. 7. Example flow cytometry analysis of CHO cells transduced with a PD-1 expression cassette either non-induced or induced with doxycyclin labelled against its N-terminal Myc tag with an anti-Myc-FITC antibody.

FIG. 8. A lentiviral payload vector consisting of an scFV expression cassette flanked by lentiviral LTRs.

FIGS. 9A, 9B and 9C. Flow cytometry density plots of three rounds of MACS selection against CHO-PD1 cells transduced with a spiked scFv library. Cells were labelled with anti-HA-PE. The plots show an increase in the numbers of HA positive cells at the end of each round.

FIGS. 10A and 10B. Flow cytometry plots showing the cell sort for downstream genomic DNA isolation and NGS analysis. FIG. 10A shows density plots showing profile of CHO cells cultured in the absence of doxycycline (left panels) or after induction with doxycycline for cell sorting (right panels) stained with anti-HA-PE (X-axis) for the presence of bound scFv and anti-Myc-FITC (Y-axis) for the presence of PD-1. FIG. 10B shows FACS plot zoomed in on the cell sorting gate. Thick line squares highlight cells double positive for the PD-1 target and HA tag.

FIG. 11A shows a schematic diagram of scFv binding to EpCAM generating FRET; and FIG. 11B shows associated FACS data.

FIG. 12. Schematic diagram showing sequestration of secreted scFvs by “capturing” CHO cells labelled with intracellular dye.

FIG. 13. FACS analysis of transduced (“capturing”) CHO cells co-expressing a target antigen and secreted scFv which binds the same antigen.

FIGS. 14A(i), 14A(ii), 14A(iii) and 14B. EpCAM binding analysis of selected anti-EpCAM sequences that had been re-cloned and expressed either as scFv or as whole IgG1. FIGS. 14A(i), 14A(ii) and 14A(iii) show flow cytometry analysis of binding of both scFv (left) and whole IgG (right) to CHO-EpCAM cells and CHO-X control cells. FIG. 14B shows single-cycle kinetics SPR sensorgrams of antibodies formatted as whole IgG1 and captured on protein A sensor chips binding to EpCAM-ECD. The data from the curve-fitting is shown in Table 1. SPR traces show representative data from four independent experiments.

FIGS. 15A to 15E. Assessment of antigen specificity of CAR-T cells generated with the three selected antibodies. FIG. 15A shows induction of activation marker CD25 on PBMC derived CAR-T cells in co-culture with CHO-EpCAM or CHO-X cells (co-cultured at a ratio of 5:1) as measured by flow cytometry after 48 hours of culture. FIG. 15B shows cytotoxicity of PBMC derived CAR-T cells when co-cultured with CHO-X or CHO-EpCAM as assessed by release of LDH after 48 hours of culture. xCELLigence analysis of CHO-X (FIG. 15C) or CHO-EpCAM (FIG. 15D) cells co-cultured with PBMC derived CAR-T cells (at a ratio of 1:5) monitored over 120 hours. Data was measured in three biological triplicates where the mean is represented in solid line with the standard error shown by the dotted line of the same colour. Statistical analysis was performed by two-way ANOVA with Bonferroni post-hoc tests and significance were assessed versus T cell cultured alone or CHO cells (*** P<0.001). FIG. 15E shows assessment of Jurkat CAR-T cell activation by flow cytometry analysis of induction of activation marker CD69 after 4 hours of co-culture with CHO-EpCAM, MCF7 or CHO-X cells (at a ratio of 1:1). Graph shows representative data from three independent experiments and shows the mean and SEM. Statistical analysis was performed by two-way ANOVA using Tukey's multiple comparison test for CHO-EpCAM or MCF7 cells compared to CHO-X cells (**** P<0.0001).

### DETAILED DESCRIPTION OF THE INVENTION

In one embodiment, the invention provides a method of identifying a cell which produces a specific binding partner which binds to a target polypeptide, the method comprising the steps:
(a) expressing a library of binding partners in a population of mammalian cells,
wherein each binding partner comprises a framework and a plurality of variable regions, each plurality of variable regions endowing that binding partner with a specific binding affinity for a target,
wherein each cell in the population of mammalian cells expresses at least one member of the binding partner library from one or more retroviral (e.g. lentiviral) vectors which have been integrated into the genome of that cell,
wherein each binding partner comprises a detectable tag,
wherein each binding partner is secreted from the cell in which it is produced, and
wherein each cell in the population of mammalian cells comprises an expression construct comprising a promoter (preferably an inducible promoter) operably linked to a nucleotide sequence encoding the target polypeptide, wherein the expression construct is integrated into the genome of each cell;
(b) optionally, removing cells from the population of mammalian cells to which binding partners bind;
(c) expressing (preferably inducing expression of) the target polypeptide from the expression construct such that the target polypeptide is displayed on the outer surface of each cell in the population of mammalian cells; and
(d) isolating or identifying cells within the population of mammalian cells to which specific binding partners are bound,
wherein cells to which specific binding partners are bound are isolated or identified using:
   (i) flow cytometry, or
   (ii) magnetic sorting, or
   (iii) a micro-fluidics system wherein cells from the population of mammalian cells are contained within each chamber in a plurality of isolated chambers,
      wherein the cells to which specific binding partners are bound are ones which produce specific binding partners which bind to the target polypeptide.

Preferably, the retrovirus is a lentivirus.

Preferably, the method additionally comprises, prior to Step (a), the step:
integrating, into the genome of each (or substantially each) cell in the population of cells, an expression construct comprising a promoter (preferably an inducible promoter) operably linked to a nucleotide sequence encoding the target polypeptide.

Preferably, the method additionally comprises, prior to Step (a), the step of:
contacting the population of mammalian cells with a library of retroviral particles, each particle comprising a retroviral vector encoding a member of a binding partner library and a detectable tag, under conditions such that at least one retroviral vector is integrated into the genome of each cell (or substantially each cell) in the library.

The method of the invention may additionally comprise the following step, which may be repeated one or more times, before or after Step (d), or as part of Step (d):
removing cells from the population of cells to which binding partners bind, wherein the binding partners are binding to polypeptides other than the target polypeptide.

Preferably, in Step (d), the flow cytometry is FACS or the magnetic sorting is MACS.

Preferably, in the micro-fluidics systems of Step (d), the isolated chambers are aqueous droplets or isolated pens on a solid substrate.

Preferably, the method additionally comprises the step:
(e) sequencing (part of all of) the nucleotide sequences in the isolated cells which encode the specific binding partners which bind to the target polypeptide.

In yet a further embodiment, the invention provides a process for producing a population of mammalian cells, the process comprising the steps:
(A) integrating, into the genome of each (or substantially each) cell in the population of mammalian cells, an expression construct comprising a promoter (preferably an inducible promoter) operably linked to a nucleotide sequence encoding a target polypeptide;
and
(B) contacting the population of mammalian cells with a library of retroviral particles, each particle comprising a retroviral vector encoding a member of a binding partner library and a detectable tag;
   so as to produce a population of mammalian cells wherein each cell (or substantially each cell) in the population of mammalian cells secretes one or more binding partners (e.g. antibodies or antibody mimetics), and
   wherein each cell in the population of mammalian cells displays or is capable of displaying (preferably upon induction) the target polypeptide on the outer surface of the cell.

Steps (A) and (B) may be carried out in either order.

Preferably, each cell (or substantially each cell) in the population of cells secretes or is capable of secreting 1-3, 1-2 or most preferably just 1 binding partner.

The method of the invention will, in general, be carried out in vitro or ex vivo.

Each cell (or substantially each cell) in the population of mammalian cells displays the target polypeptide on the outer surface of the cell. The target polypeptide is not secreted into the media surrounding the cell; the target polypeptide remains bound to the cell.

Generally, each cell (or substantially each cell) in the population of cells will display the same target polypeptide.

The target polypeptide preferably comprises one or more transmembrane domains in order to locate the target polypeptide in the outer cell membrane of the cell. In one embodiment, the target polypeptide is an integrated membrane protein. Preferably, it is integrated directly in the outer membrane of the cell.

In one embodiment, the target polypeptide is a fusion polypeptide which comprises an antigenic polypeptide linked to a transmembrane domain (e.g. a platelet derived growth factor receptor domain). The transmembrane domain anchors the antigenic polypeptide in the cell membrane and allows the antigenic domain to be displayed. The amino acid sequence of the antigenic polypeptide and the transmembrane domain may be linked by a short amino acid linker, e.g. 1-10 or 1-20 amino acids.

The target polypeptide may additionally comprise a detectable tag (e.g. as exemplified below), preferably a Myc epitope.

The target polypeptide may be a glycosylated polypeptide or a non-glycosylated polypeptide.

In some embodiments, the target polypeptide is a single-pass membrane protein or a multiple-pass membrane protein. In some embodiments, the target polypeptide comprises 1, 2, 3, 4, 5, 6, or 7 transmembrane domains.

In some embodiments, the target polypeptide is a G-protein coupled receptor (GPCR) (e.g. DRD1). In some embodiments, the target polypeptide is an immunotherapy target, e.g. CD19, CD40 or CD38. In some embodiments, the target polypeptide is a protein which increases/decreases proliferation of cells, e.g. a growth factor receptor. In some embodiments, the target polypeptide is an ion channel polypeptide.

In some preferred embodiments, the target polypeptide is an immune checkpoint molecule. Preferably, the immune checkpoint molecule is a member of the tumour necrosis factor (TNF) receptor superfamily (e.g. CD27, CD40, OX40, GITR or CD137) or a member of the B7-CD28 superfamily (e.g. CD28, CTLA4 or ICOS). Preferably, the immune checkpoint molecule is PD1, PDL1, CTLA4, Lag1 or GITR.

In some embodiments, the target polypeptide is not avidin or streptavidin.

In other embodiments, the target polypeptide is displayed on the outer surface of the cell in a target polypeptide/MHC1 complex. In such an embodiment, the target polypeptide and the MHC1 may be both over-expressed within the cells in order to achieve presentation of the target polypeptide in the MHC groove.

Each cell in the population of mammalian cells comprises an expression construct comprising a promoter (preferably an inducible promoter) operably linked to a nucleotide sequence encoding the target polypeptide. The expression construct is integrated into the genome of each cell.

Nucleotide sequences encoding the same target polypeptide will be integrated into all or essentially all of the cells in the population of mammalian cells.

The expression construct may be integrated into the genomes of each cell by any suitable means.

Preferably, the expression construct is not integrated in the genome of each cell using a viral vector.

Examples of suitable integration methods include the use of CRE-LoxP recombination, FLP-FRT recombination, CRISPR-based homology directed recombination, TALENs, Meganuclease and methods using Zinc-finger polypeptides.

Cre/Lox and Flp/Frt both require that specific recombination sites, which are recognised by the recombinase, have previously been inserted into the cell genome to create a landing pad. These recombination sites remain after the recombination event, so a footprint is left behind in the cell genome.

Preferably, the expression construct is integrated into the genomes of each cell by using Flp/FRT.

In some embodiments of the invention, the method additionally comprises the step:
integrating, into the genome of each (or substantially each) cell in the population of cells, an expression construct comprising a promoter (preferably an inducible promoter) operably linked to a nucleotide sequence encoding the target polypeptide.

The expression construct comprises a promoter (preferably an inducible promoter) element. The promoter may, for example, be a SFFV, CMV, SV40, EF1-Aplha, PGK, E1A, Ubiquitin, Chicken Beta Actin or RSV promoter.

Preferably, the inducible promoter element comprises a DNA sequence capable of binding proteins that can form a basal transcription complex and initiate transcription and a plurality of Tet operator sequences to which the Tet repressor protein (TetR) is capable of binding. In this bound state, tight suppression of transcription is obtained. However, in the presence of doxycycline, suppression is alleviated, thus allowing the promoter to gain full transcriptional activity. Such an inducible promoter element is preferably placed downstream of another promoter, e.g. the CMV promoter, or one of the other aforementioned promoters.

The expression construct preferably comprises a suitable signal polypeptide which directs the target polypeptide to the outer cell membrane.

Examples of suitable signal polypeptides include those from: BM-40 (osteonectin SPARC), Vesicular Stomatitis Virus G (VSVG) protein, chymotrypsinogen, human interleukin-2 (IL-2), Gaussia luciferase, human serum albumin, influenza haemagglutinin, human insulin and immunoglobulin genes.

In some embodiments, the cells comprise multiple copies of the target polypeptide expression construct in order to increase the levels of target polypeptide which are expressed; preferably, these are each integrated into the cell genome. Increasing levels of target polypeptide expression may also be achieved by increasing the time of cell culturing.

The target polypeptide expression construct may also comprise an antibiotic resistance gene, e.g. one encoding resistance to puromycin.

In a particularly preferred embodiment of the invention, an anti-apoptosis factor is inserted after (i.e. 3′) an IRES downstream (i.e. 3′) of the last stop codon of the nucleic acid encoding the target polypeptide. This provides a configuration where a promoter initiating transcription is upstream (i.e. 5′) to the coding sequence of the target polypeptide gene which is then followed (3′) by an IRES, which is then followed (3′) by the coding region for the anti-apoptosis factor gene. In this configuration, both the target polypeptide and anti-apoptosis factor are encoded by the same mRNA, but due to the relatively low efficiency of IRES-mediated translation, the target polypeptide will be translated in greater abundance than the anti-apoptosis factor.

The target polypeptide is displayed on a population of mammalian cells. The cells may be isolated cells, e.g. they are not present in a living animal.

Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells. Preferred human cells include HEK293, HEK293T, HEK293A, PerC6, 911 and HeLa cells. Other preferred cells include CHO, VERO and COS cells. Most preferably, the cells are CHO cells.

Preferably, all or substantially all of the cells in the population display the target polypeptide. Preferably, all or substantially all cells in the population express less than or less than 5, more preferably 1, 2 or 3, and most preferably a single binding partner.

In the method of the invention, a library of binding partners is expressed in the population of mammalian cells. The aim is to identify at least one specific binding partner which binds to an exposed region or domain of the target polypeptide in such a way that cells to which such specific binding partners are bound can be identified and/or isolated.

As used herein, the term “specific binding partner” relates to the ability of the binding partner to bind to the target polypeptide with a desired degree of specificity and/or affinity. The specific binding partner might not bind exclusively to the target polypeptide. Preferably, a specific binding partner specifically binds if its affinity for its target polypeptide is about 5-fold greater than its affinity for a non-target polypeptide. Ideally, there is no significant cross-reaction or cross-binding with undesired substances.

The affinity of the specific binding partner may, for example, be at least about 5 fold, such as 10 fold, such as 25-fold, especially 50-fold, and particularly 100-fold or more, greater for a target molecule than its affinity for a non-target polypeptide.

In some embodiments, binding between a specific binding partner and a target polypeptide means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Binding partners may, for example, bind with an EC₅₀ of 50 nM or less, 10 nM or less, 1 nM or less, 100 pM or less, or more preferably 10 pM or less. The term “EC₅₀” as used herein, is intended to refer to the potency of a compound by quantifying the concentration that leads to 50% maximal response/effect. EC₅₀ may be determined by Scatchard, FACS, ELISA or biological potency assay, for example.

Each binding partner comprises a framework and a plurality of variable regions, each plurality of variable regions endowing that binding partner with a specific binding affinity for a target. The core framework may comprise one or more polypeptides. Preferably, there are 2-10, more preferably 2-6, 3-6, 4-6 or 5-6 variable regions. The binding partners will, in general, be polypeptides. These may or may not be glycosytated. The binding partners may be viewed as potential binding partners or potential specific binding partners of the target polypeptide.

The binding partners (e.g. antibodies or antibody mimetics) are secreted by or secreted from or secreted out of the cells from which they are produced. In some embodiments, the binding partners are secreted out of the cells from which they are produced and into the medium which surrounds the cells. In other terms, in this embodiment, the binding partners are released from the cells.

In other embodiments, the binding partners are secreted from the cells from which they are produced. The binding partners may or may not then be released into the medium which surrounds the cells.

In some embodiments, secretion may be aided by the inclusion of an N-terminal-signal polypeptide. Examples of suitable signal polypeptides include those from: BM-40 (osteonectin SPARC), Vesicular Stomatitis Virus G (VSVG) protein, chymotrypsinogen, human interleukin-2 (IL-2), Gaussia luciferase, human serum albumin, influenza haemagglutinin, human insulin, and immunoglobulin genes. In some preferred embodiments, the signal polypeptide is an immunoglobulin light chain signal sequence.

Binding partners and the target polypeptide will be synthesized in the mammalian cells by ribosomes which are attached to the rough endoplasmic reticulum (ER). These polypeptides will both comprise signal peptides in order to direct the passage of the polypeptides to the cell's secretory pathway. After they are synthesized, these polypeptides will translocate into the ER lumen, where they may be glycosylated and where molecular chaperones aid protein folding. Vesicles containing the polypeptides then enter the Golgi apparatus. In the Golgi apparatus, any glycosylation of the polypeptides may be modified and further posttranslational modifications, including cleavage and functionalization, may occur. The polypeptides are then moved into secretory vesicles which travel along the cytoskeleton to the edge of the mammalian cell. Further modification may occur in the secretory vesicles. Eventually, there is vesicle fusion with the cell membrane at a structure called the porosome, in a process called exocytosis, which results in release of the contents of the vesicle to the surrounding medium. Membrane-integrated proteins will be retained in the cell's plasma membrane when the vesicle contents are shed.

Since both binding partners and target polypeptides are produced via this secretory pathway, it is possible that binding of some binding partners to the target polypeptide will occur during the course of this pathway. If this is the case, then the binding partner will not be secreted out of the cell into the external medium; it will remain bound to the target polypeptide. The binding partner and target polypeptide will therefore be presented—together—on the outer surface of the cell.

In some embodiments, the binding partners are secreted from the cells in which they are produced in a form wherein their CDR sequences are bound to the target polypeptide. In other embodiments, the binding partners are secreted from the cells in which they are produced in a form wherein their CDR sequences are not bound to the target polypeptide.

The binding partners are not covalently attached, directly or indirectly, to the surface of the cells. The binding partners are free to diffuse within the medium surrounding the cells (apart from those binding partners which bind to the target polypeptide within the secretory pathway).

In some preferred embodiments, the binding partner is an antibody or an antibody mimetic. In this embodiment, Step (a) comprises, inter alia, expressing a library of antibodies or antibody mimetics in a population of mammalian cells.

An “antibody” is an immunoglobulin molecule which is capable of specific binding to a target, such as a carbohydrate, polynucleotide, lipid, polypeptide, etc., through at least one antigen recognition site, located in the variable region of the immunoglobulin molecule.

“Antibody” as used herein includes a wide variety of structures, as will be appreciated by those in the art, that in some embodiments contain at a minimum a set of 6 CDRs, including, but not limited to traditional antibodies (including monoclonal antibodies), humanized and/or chimeric antibodies, antibody fragments, engineered antibodies, multi-specific antibodies (including bi-specific antibodies), and other analogues known in the art.

Preferably, the antibody comprises at least one VH domain and at least one VL domain. The VH domain and VL domain may be from different species, e.g. a chimeric antibody and/or a humanized antibody. That is, the CDR sets can be used with framework and constant regions other than those from which they were originally obtained.

In some embodiments, the antibody can be a mixture from different species, e.g. a chimeric antibody and/or a humanized antibody. That is, the CDR sets can be used with framework and constant regions other than those from which they were originally obtained.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs.

In one embodiment, the antibody is an antibody fragment. Specific antibody fragments include, but are not limited to, (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al., 1989, Nature 341:544-546) which consists of a single variable region; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883); (viii) bispecific single chain Fv (e.g. WO 03/11161); and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). The term “antibody” also includes domain antibodies, Nanobodies and UniBodies.

The term “antibody” also includes fusion proteins comprising an antibody portion or fragment with an antigen recognition site. Most preferably, the antibody is a scFv antibody.

The antibody library may, for example, comprise immunoglobulin polypeptides of a certain type or class. For example, the library might encode antibody μ, γ1, γ2, γ3, γ4, α1, α2, ε, or δ heavy chains, and/or antibody K or A light chains. The antibody isotypes may be IgM, IgD, IgG, IgA or IgE. Preferably, the antibodies are IgG1, IgG2, IgG3 or IgG4.

Although each member of any one library described herein may encode the same heavy or light chain constant region, the library may collectively comprise at least 10⁶, 10⁷, 10⁸, 10⁹, 10¹⁰, 10¹¹, 10¹², 10¹³, 10¹⁴ or 10¹⁵ or more different variable regions, i.e., a “plurality” of variable regions associated with a common constant region.

In a particularly-preferred embodiment of the invention, the binding partner is a scFv antibody.

The scFv antibodies in the library may also have variations in the framework regions (e.g. variations in the heavy and/or light chain constant regions), and/or variations the variable regions (e.g. VH, VL or one or more of the CDRs).

The methods of the invention are not limited to methods involving antibodies. They may also be practised through the use of antibody mimetics. A wide variety of antibody mimetic technologies are known in the art. In particular, technologies such as Affibodies, DARPins, Anticalins, Avimers, and Versabodies that employ binding structures that, while they mimic traditional antibody binding, are generated from and function via distinct mechanisms.

Affibody molecules represent a new class of affinity proteins based on a 58-amino acid residue protein domain, derived from one of the IgG-binding domains of staphylococcal protein A. This three-helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren PA, Binding proteins selected from combinatorial libraries of an α-helical bacterial receptor domain, Nat Biotechnol 1997; 15:772-7. Ronmark J, Gronlund H, Uhlen M, Nygren PA, Human immunoglobulin A (IgA)-specific ligands from combinatorial engineering of protein A, Eur J Biochem 2002; 269:2647-55.). Further details of Affibodies and methods of production thereof may be obtained by reference to U.S. Pat. No. 5,831,012.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins are ubiquitous binding molecules, which occur, unlike antibodies, intra- and extra-cellularly. Their unique modular architecture features repeating structural units (repeats) which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains. Additional information regarding DARPins and other DRP technologies can be found in US 2004/0132028 and WO 02/20565.

Anticalins are an additional antibody mimetic technology. However, in this case, the binding specificity is derived from lipocalins, a family of low molecular weight proteins that are naturally and abundantly expressed in human tissues and body fluids.

Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains. Additional information regarding Anticalins can be found in U.S. Pat. No. 7,250,297 and WO 99/16873.

Another antibody mimetic technology useful in the context of the instant invention is Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets. Additional information regarding Avimers can be found in US 2006/0286603, 2006/0234299, 2006/0223114, 2006/0177831, 2006/0008844, 2005/0221384, 2005/0164301, 2005/0089932, 2005/0053973, 2005/0048512, and 2004/0175756.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

Given the structure of Versabodies, these antibody mimetics offer a versatile format that includes multi-valency, multi-specificity, a diversity of half-life mechanisms, tissue targeting modules and the absence of the antibody Fc region. Additional information regarding Versabodies can be found in US 2007/0191272.

In other embodiments, the binding partner is not an antibody or antibody mimetic.

In some embodiments, the binding partner is a T-cell receptor, preferably a soluble T-cell receptor.

For example, the desired specific binding partner of the target polypeptide may be a polypeptide ligand to which the target polypeptide is capable of binding. In such cases, the library of binding partners may be library of polypeptides whose amino acid sequences are based upon the amino acid sequence of a polypeptide ligand which is known to bind to the target polypeptide. For example, the polypeptides in such a library may have at least 60%, 70%, 80%, 90% or 95% amino acid sequence identity with the known polypeptide ligand.

As used herein, the term “library” refers to a plurality of (potential) binding partners, each having a different binding specificity and/or affinity. Each binding partner has a framework (which may or may not be common to all binding partners), and a plurality of different variable regions. For example, the members of the binding partner library may vary in the CDR sequences of one or more of their CDRs.

The plurality of binding partners will, in general, be encoded by a plurality of polynucleotides.

In certain embodiments, the library of binding partners may comprise at least 10⁶, 10⁷, 10⁸, 10⁹, 10¹⁰, 10¹¹, 10¹², 10¹³, 10¹⁴ or 10¹⁵ or more different binding partners.

In some embodiments, the different binding partners in the library are related through, for example, their origin from a single animal species (for example, human, mouse, rabbit, goat, horse), tissue type, organ or cell type.

In other embodiments, the library is a library of naturally-occurring polypeptides, which might be enriched. In yet other embodiments, the library is a library of synthetic polypeptides.

Each binding partner comprises a detectable tag. The tag may be an affinity tag.

The function of the tag is to aid identification and/or isolation of the cells to which specific binding partners are bound.

The tag is preferably a polypeptide tag, i.e. the binding partner comprises a fusion polypeptide wherein amino acid sequence of the binding partner and tag are contiguous or joined by a peptide linker (e.g. 1-15 amino acids).

The polypeptide tag may be joined at the N- or C-terminus of the binding partner, preferably at the C-terminus.

Examples of suitable tags include influenza haemagglutinin peptide, T7 gene 10 peptide, bacteriophage V5 epitope, BPV E2 epitope, FLAG tag, Histidine affinity tag, poly-histidine tag comprising 6, 8 or 10 or more residues, HSV epitope tag, Myc epitope, Protein C epitope, HBV Si epitope, Streptavidin-binding protein, Strep-tag, C-terminal residues of VSV protein G or any other peptide sequence that can be recognised with high affinity and specificity by antibodies or other proteins or systems.

The binding partner could also be tagged by fusion to a fluorescent protein to allow direct detection on the cell surface (e.g. GFP, RFP, YFP, BFP or any other protein that emits a fluorescent signal upon specific excitation) or an enzyme that can generate a coloured reaction product (e.g. alkaline phosphatase, horseradish peroxidase) or enzyme that generates a light signal (e.g. luciferases from Metridia species, Renidla species, fire-flies or bacteria).

In some embodiments, the tag comprises a functional domain whose presence and/or activity may be established and/or quantified. Examples of such functional domains include domains which promote or inhibit cell proliferation (e.g. appropriate domains of growth factors).

In some embodiments (e.g. wherein the flow cytometry is FACS), the tag is preferably a polypeptide tag (e.g. as listed above) to which an antibody is capable of binding with high affinity and specificity, such antibody preferably being covalently attached to a fluorophore that emits a specific light wavelength after excitation by a laser using light of a different wavelength (e.g. FITC, phycoerythrin, APC, PerCP, DyLight of various wavelengths and the Alexa series of fluorophores).

Most preferably, the tag is an HA tag or a Myc tag, and/or the detection antibody is linked to FITC, PE, or an Alexa or Dylight fluorophore.

In other embodiments (e.g. wherein the magnetic sorting is MACS), the tag is preferably a polypeptide antigen, to which an antibody may be bound with high affinity and specificity (as listed above), such antibody being preferably covalently linked to paramagnetic particles (e.g. Dynabeads (Thermofisher Scientific, Loughborough, UK), or MACS microbeads (Miltenyi Biotec, Bisley, UK)) such that cells labelled with the paramagnetic beads can be attracted to a magnet. Most preferably the tag is an HA tag or a Myc tag.

Preferably, the retroviral vector additionally comprises a nucleotide sequence encoding the tag, i.e. the binding partner and tag are expressed as a fusion polypeptide.

One or more different detectable tags may be used, i.e. it is not necessary for all of the binding partners in the library to comprise the same tag. For procedural efficiency, however, it is preferable that they all have the same tag.

The above comments apply also to the target polypeptide's detectable tag, mutatis mutandis

Each cell in the population of mammalian cells expresses at least one member of the binding partner library from one or more retroviral vectors which have been integrated into the genome of that cell.

For example, each cell in the population of mammalian cells may express 1, 2, 3, 4 or 5 members of the binding partner library from 1, 2, 3, 4 or 5 retroviral vectors which have been integrated into the genome of that cell.

Preferably, each cell in the population of mammalian cells expresses only one member of the binding partner library from a retroviral vector which has been integrated into the genome of that cell.

Retroviruses will preferably have the ability to integrate their genome, or modified versions of, into the chromosomes of both dividing and non-dividing cells. Retroviruses cover members of the Gamma retroviral family preferably, which will include Murine-Leukaemia virus (MLV), Feline Leukaemia virus (FLV), or Moloney Murine Leukaemia Virus (MMLV), or members of the Alpha retrovirus family such as Rous-Sarcoma virus (RSV).

Retroviral vectors may also include genetically enhanced retroviral vectors such as Murine Stem Cell Virus (MSCV).

Most preferably, the retroviral vector will be a lentiviral vector capable of infecting non-dividing and dividing cells.

Lentiviruses are a subset of the retroviridae family that are increasingly being used for transgene delivery and protein expression, particularly in progenitor cell populations such as haematopoietic stem cells and T cells. Unlike most retroviruses, lentiviruses are able to deliver their genome, or modified forms thereof, independent of the cell cycle, and often achieve higher efficiency of cellular infection in a shorter time frame. This makes them a much more effective viral vector for both research and clinical use.

The lentivirus family consists of 10 viruses at present. These species are divided into five groups including Bovine lentivirus group (Bovine immunodeficiency virus and Jembrana disease virus), Equine lentivirus group (Equine infectious anaemia virus, Feline lentivirus group, Feline immunodeficiency virus, Puma lentivirus), Ovine/caprine lentivirus group (Caprine arthritis encephalitis virus, Visna/maedi virus), Primate lentivirus group, (Human immunodeficiency virus 1, Human immunodeficiency virus 2, Simian immunodeficiency virus).

The retrovirus (e.g. lentivirus) must be one which is capable of infecting the mammalian cells.

In a preferred embodiment, the lentivirus is Human immunodeficiency virus 1, Simian immunodeficiency virus or Equine infectious anaemia virus.

In a more preferable embodiment, the lentivirus is Human immunodeficiency virus 1 or Equine infectious anaemia virus.

In one embodiment, the population of mammalian cells are produced by infecting an initial (homogenous) population of cells with a plurality of retroviral (e.g. lentiviral) particles encoding a binding partner library.

In some embodiments, therefore, the method of the invention additionally comprises, prior to Step (a), the step of:
contacting the population of mammalian cells with a library of retroviral (e.g. lentiviral) particles, each particle comprising a retroviral (e.g. lentiviral) vector encoding a member of a binding partner library and a detectable tag, under conditions such that at least one retroviral (e.g. lentiviral) vector is integrated into the genome of each cell (or substantially each cell) in the library.

For example, each retroviral (e.g. lentiviral) particle may comprise a retroviral (e.g. lentiviral) vector comprising a promoter (which is inducible or non-inducible), a signal peptide (to promote secretion of the binding partner), a binding partner-coding sequence and a detectable tag-coding sequence. These elements are flanked by retroviral (e.g. lentiviral) long terminal repeats (LTRs).

The promoter may be, for example, a spleen focus-forming virus (SFFV) promoter.

In order to generate virus particles, a plasmid containing such a vector construct may be co-transfected into a producer cell line with additional plasmids that provide genes for replication and encapsidation in trans.

After integration of the retroviral (e.g. lentiviral) vector into the cell genome, the retroviral (e.g. lentiviral) vector will still comprise the promoter, the signal peptide, the binding partner-coding sequence and the detectable tag-coding sequence, and the LTRs (i.e. all of the sequences between the LTRs will be incorporated into the genome of the host cell).

The retroviral (e.g. lentiviral) vector may additionally comprise a nucleotide sequence encoding an anti-apoptosis factor.

Step (b) comprises the optional step:
optionally, removing cells from the population of cells to which binding partners bind.

This step will be particularly relevant if the target polypeptide expression construct comprises an inducible promoter.

At this stage, expression of the target polypeptide is not induced. Therefore, the cells in the population of cells will not yet display the target polypeptide on their outer surface. Consequently, any binding of binding partners to the cells at this stage will not be specific for the target polypeptide.

In this way, cells to which binding partners are bound prior to the display of the target polypeptide are removed from the population of cells before the target polypeptide is expressed. This helps to reduce non-specific binding of the binding partner.

Such cells may be removed by FACS sorting where the binding to the cell surface of the binding-partner is detected using a fluorophore labelled antibody to the tag on the binding partner and the population of unstained cells is collected and the population of stained cells is removed. Alternatively, such cells are removed by MACS using paramagnetic microbeads linked to the anti-tag antibody such that non-specific binders are retained on the magnetic column and unbound cells flow through the column and are collected.

This negative selection step may be performed at any stage in the selection process: before performing positive selection, or after the first, second or third rounds of positive selection. Furthermore, any number of positive selections and/or negative selections may be done in any combination as necessary for the isolation of specific binding partners against the target polypeptide.

In particular, the method of the invention may additionally comprise the following step, which may be repeated one or more times, before or after Step (d), or as part of Step (d):
removing cells from the population of cells to which binding partners bind, wherein the binding partners are binding to polypeptides other than the target polypeptide.

Step (c) of the method of the invention comprises expressing or inducing expression of the target polypeptide from the expression construct such that the target polypeptide is displayed on the outer surface of each cell in the population of mammalian cells.

In some embodiments, the expression construct comprises an inducible promoter element.

Expression from this promoter may be obtained by inducing expression or relieving suppression of the promoter.

For example, if the promoter comprises a plurality of Tet operator sequences and the Tet repressor protein (TetR) is present in the cell, then tight suppression of transcription is obtained. However, in the presence of doxycycline, suppression is alleviated, thus allowing the promoter to gain full transcriptional activity.

Step (b) may therefore comprise the step of contacting the cells with doxycycline (which displaces the Tet repressor proteins, thus allowing the promoter to gain full transcriptional activity).

In order to reduce levels of (i) binding of secreted binding partners to non-target polypeptides on the surfaces of the mammalian cells and (ii) the binding of target polypeptide-specific binding partners to cells from which those binding partners have not been secreted, all or part of the population of mammalian cells may be contacted with (e.g. co-cultured with) an excess of cells (“capturing cells”) which express the target polypeptide but do not express a binding partner (e.g. do not express a binding partner from the library of binding partners).

In some embodiments, therefore Step (b) additionally comprises the step:
contacting all or part of the population of mammalian cells with an excess of capturing cells which express the target polypeptide (and optionally a second detectable tag) but do not express a binding partner, wherein each capturing cell comprises a label which allows the capturing cells to be distinguished from the population of mammalian cells.

Alternatively, this step is carried out in or after Step (c).

In this embodiment, the excess of capturing cells will help to remove from the culture media secreted binding partners which bind to non-target polypeptides on the surfaces of the mammalian cells. This reduces the background level of non-specific binding.

Furthermore, the excess of capturing cells will help to remove from the culture media secreted target polypeptide-specific binding partners which might otherwise bind to to cells from which those binding partners have not been secreted. This also reduces the background level of non-specific binding.

The capturing cells may or may not be of the same type as the population of mammalian cells. Preferably, the capturing cells are of the same type as the population of mammalian cells. More preferably, the capturing cells and population of mammalian cells are both CHO cells.

The capturing cells are present in an excess amount compared to the number of cells in the population of mammalian cells. Preferably, the excess is at least a 2:1 excess (capturing cells:population of mammalian cells), more preferably at least a 4:1 or 9:1 excess. In some embodiments, the excess is a 2:1-5:1 excess, 5:1-10:1 or 10:1-50:1 excess.

The capturing cells are preferably each labelled with a label (which may be the same or different) which allows them to be distinguished from the population of mammalian cells. The label will in general be different from any detectable tags which are attached to the binding partners or to the target polypeptides. For example, the capturing cells may each be labelled with a fluorescent label (e.g. a fluorescent intracellular dye). Preferably, the fluorescent label is a coumarin-based label, e.g. 7-amino-4-chloromethyl-coumarin.

Preferably, the target polypeptide is expressed in the capturing cells in the same manner as the expression of the target polypeptide in the population of mammalian cells, e.g. wherein each cell in the capturing cells comprises an expression construct comprising a promoter (preferably an inducible promoter) operably linked to a nucleotide sequence encoding the target polypeptide, wherein the expression construct is integrated into the genome of each capturing cell. Preferably, the inducer of the expression of the target polypeptide in the population of mammalian cells is the same inducer of expression of the target polypeptide in the capturing cells.

The capturing cells are contacted with (e.g. incubated with) the population of mammalian cells for a time which allows some or all secreted binding partners in the culture medium to bind to the capturing cells.

In embodiments wherein the expression construct comprises an inducible promoter, an inducer of expression of the target polypeptide (e.g. doxycycline) may be added to the culture medium at this time or in Step (c).

The cells may then be harvested and stained with (different) antibodies against the detectable tag on the binding partner and the (different) detectable tag on the target polypeptide. For example, anti-Myc-FITC and anti-HA-PE antibodies may be used or, for FRET analysis, anti-HA-PE and anti-Myc-AlexaFluor647 antibodies may be used.

Preferably, the fluorescence emission profile of the capturing cells' label does not overlap with any of the fluorescently-labelled antibodies against the detectable tags; and therefore the level of the fluorescence emission of the capturing cells' label can be used to distinguish the capturing cells from the population of mammalian cells. In this way (or any other suitable method), the capturing cells may be separated from the population of mammalian cells. The capturing cells may then be discarded.

The capturing cells may be separated from the population of mammalian cells at this time or in Step (c) or Step (d), preferably in Step (d).

Preferably, the capturing cells are separated from the population of mammalian cells using flow cytometry, e.g. FACS.

Step (d) of the method of the invention comprises identifying and/or isolating cells within the population of mammalian cells to which specific binding partner are bound. Specific binding partners are those which bind to the target polypeptides which are displayed on those cells. The cells to which specific binding partners are bound are ones which produce specific binding partners against the target polypeptide. In this way, specific binding partners against the target polypeptide can be identified and/or isolated.

The cells to which specific binding partners are bound are isolated or identified using:
(i) flow cytometry, or
(ii) magnetic sorting, or
(iii) a micro-fluidics system wherein cells from the population of cells are contained within a plurality of isolated chambers.

In flow cytometry, the cells are sorted using an automatic cell sorter on the basis of whether or not specific binding partners are bound to the cells.

In one embodiment, the flow cytometry is fluorescence activated cell sorting (FACS); the detectable tag which is attached to the specific binding partner is a polypeptide tag as disclosed herein; and the polypeptide tag is detected using a fluorophore-labelled secondary antibody.

In another embodiment, the magnetic sorting is magnetic activated cell sorting (MACS); the detectable tag which is attached to the specific binding partner is a polypeptide tag as disclosed herein; and the polypeptide tag is detected using a paramagnetic particle-labelled secondary antibody. Magnetic nanoparticles are well known in the art (e.g. from Miltenyi Biotec).

In a further and particularly-preferred embodiment, the cells to which specific binding partners are bound are isolated or identified using a micro-fluidics system. In such a system, cells from the population of mammalian cells are contained (and maintained) within a plurality of isolated chambers. One key advantage of such systems is that each cell (or a small number of cells) can be assayed for the production of a specific binding partner in isolation from other cells (or the majority of the other cells). This reduces the problem of cross-labelling.

Microfluidics deals with the behaviour, precise control and manipulation of fluids that are geometrically constrained to a small, typically sub-millimetre, scale.

In the method of the invention, the cells from the population of mammalian cells are contained within a plurality of isolated chambers (e.g. discrete or isolated units). The chambers may, for example, be droplets (e.g. aqueous droplets) within a stabilised oil/water emulsion (stabilised so that the aqueous droplets do not fuse) or pens on the surface of a solid substrate.

In some embodiments, each chamber may comprise 1, 1-2, 1-5, 1-10, 5-10, 10-20, 20-30, 30-40, 40-50, 50-100 or 100-500 cells. In some preferred embodiments, each chamber comprises 30-50 cells. In other preferred embodiments, each chamber comprises 1 cell.

In some embodiments, each chamber may have a fluid (e.g. liquid) volume of 1-2000 pL, preferably 100-1000 pL and more preferably about 200 pL. For example, the droplets may be about 200 pL; and pens may be 1750 pL, 750 pL, 500 pL, 250 pL or 1 nL.

Within each chamber, the necessary detection reagents are also included such that each cell can be assayed for the production of a target polypeptide-specific binding partner. For example, each chamber may also comprise a labelled secondary antibody against the detectable tag (e.g. a fluorophore-labelled secondary antibody).

The chamber may also comprise physiologically-acceptable aqueous composition, e.g. cell culture medium.

In one embodiment, the chambers are droplets comprising an aqueous liquid which can be passed (preferably at high speed) through microfluidic channels for analysis by lasers for specific fluorescence. The detection of such fluorescence indicates cell surface labelling of the target polypeptide by a fluorescence-labelled specific binding partner. Positive droplets may then be isolated and dispensed (e.g. into microtitre plates) for recovery and expansion of positive individual cells. An example of such an instrument is the Cyto-Mine® manufactured by Sphere Fluidics (Cambridge, UK).

In another embodiment of the invention, the chambers are pens which are arranged in an array on a solid substrate (e.g. a chip). In this case, the solid substrate may comprise a plurality of pens (e.g. discrete locations) distributed over the surface of the substrate. The pens may be open-ended chambers which are arrayed horizontally on a chip adjacent to flow channels.

For example, the chip might comprise 1000-20000 pens, more preferably 1500-15000 pens, and most preferably about 1750, 3500 or 14000 pens (most preferably with a volume of 1700 pL, 750 pL and 250 pL respectively).

Opto-fluidic technology (e.g. that uses light and millions of light-actuated pixels) may be used to move individual cells into the chambers (pens). In this embodiment also, cells can be assayed in isolation (or in small numbers) in the chambers for desired properties using fluorescence imaging of each pen. An example of such an instrument is the Beacon platform produced by Berkeley Ughts (Emeryville, Calif.).

Both of these systems work by detecting the bound fluorophore which will be concentrated at the surface of the cell; this will give a stronger signal than unbound fluorophore that is distributed throughout the culture medium and therefore gives a diffuse signal. Preferably, the detectable tag is an HA tag and an antibody linked to FITC or PE is used to detect the HA tag.

In some embodiments of the invention, Step (d) comprises isolating or identifying cells or groups of cells to which specific binding partners are bound by passing the population of cells through a flow cytometry or microfluidics system more than once (e.g. 2, 3, 4 or 5 times). In subsequent passes, the number of cells per chamber may be reduced until individual specific binding partner expressing cells are isolated or identified.

For example, in the first pass, each chamber may comprise up to about 10, 20, 30, 40 or 50 cells per chamber (preferably 30 to 50 cells per chamber); and in the second pass, each chamber may comprise 1-2, preferably 1, cell per chamber in order to identify cells expressing specific binding partners.

Furthermore, these platforms may be used to screen out non-specific binding partners (i.e. binding partners that bind non-specifically to cells (e.g. to CHO cells) or bind to other cells on the cell surface) by incorporating non-target expressing cells into the system. In this case, each chamber contains a cell expressing a binding partner that is mixed with a multiple of cells that do not express the target polypeptide. In this case, if all of the cells in the chamber are labelled by the binding partner, then this indicates non-specific binding, whereas if only one cell is labelled, then this indicates specific binding. Cells can then be recovered from the chamber and then re-screened at one cell per chamber to select the cell expressing the specific binding partner.

Also provided therefore is a method of the invention, wherein each chamber comprises one cell which expresses the target polypeptide and a plurality (e.g. 1-50) of cells (e.g. CHO cells) which do not express the target polypeptide; and wherein chambers in which cell surface binding has been detected are rejected if cell surface binding is detected on more than one cell per chamber. Preferably, a fluorophore-labelled second antibody is used to detect the detectable tag.

The Beacon system additionally provides the ability to identify positive cells at one cell per pen, which can then be assayed directly for non-specific binding by flowing non-target polypeptide expressing cells (e.g. CHO cells) through the chip such that multiple CHO cells are present at the mouth of each pen, but not directly in the pen. Secreted scFv by the cell contained within the pen will diffuse to the mouth of the pen. Labelling of these CHO cells at the mouth of a pen indicates non-specific binding. Subsequently target polypeptide-positive cells can be flowed through the chip to confirm the location of pens containing cells that produce specific binding members that do not non-specifically label CHO cells. After flushing the chip, these cells can then be recovered and expanded.

Either of these platforms can be integrated into a workflow in combination with MACS or FACS selection.

Also provided therefore is a method of the invention, wherein a micro-fluidics system is used wherein one or more cells from the population of mammalian cells are contained within each pen in an array of isolated pens; wherein non-target polypeptide displaying cells (e.g. CHO cells) are contacted with or juxtaposed against the edges of the isolated pens in the array such that binding partners which are secreted from the cells within the pens are capable of contacting the non-target polypeptide displaying cells; and rejecting pens in which cell surface binding has been detected on the surface of the non-target polypeptide displaying cells.

It should be understood by those skilled in the art that the Cyto-Mine® and Beacon instruments are discussed herein to demonstrate the general applicability of microfluidics systems to the identification of specific binding partners using methods of the current invention; equally any high-throughput detection system that allows individual cells to be screened in an isolated reaction chamber may be used.

In some embodiments of the invention, the detectable tag is detected using a fluorophore-labelled secondary antibody against the detectable tag, wherein the fluorophore is one member of a donor-acceptor FRET (fluorescence resonance energy transfer) pair. The other member of the donor-acceptor FRET pair may be attached to an antibody against a detectable tag (e.g. Myc epitope) on the target polypeptide.

(FRET) is a physical phenomenon in which a donor fluorophore in its excited state non-radiatively transfers its excitation energy to a neighbouring acceptor fluorophore, thereby causing the acceptor to emit its characteristic fluorescence.

To increase the specificity of the target polypeptide-binding partner signal by secreted binding partners (e.g. scFvs), a FRET signal may be used between an acceptor and donor fluorophore which co-bind to the target polypeptide-binding partner complex. The target polypeptide may be bound by a FRET acceptor antibody which binds to a second detectable tag (for example a Myc-tag, HIS-tag, FLAG-tag, etc.) which is part of the target polypeptide (e.g. fused to it), or which binds to the target polypeptide directly. The FRET donor antibody may bind to a first (different) detectable tag (e.g. HA tag or alternative affinity tag) on the binding partner (e.g. scFv). The roles of the FRET acceptor and donor antibodies may, of course, be reversed. If the secreted binding partner (e.g. scFv) binds to the target polypeptide, then the acceptor/donor pair are brought within the Förster distance and they then generate a FRET emission from the acceptor fluorophore, once the donor has been excited. This signal may then be utilised in a FACS experiment, for example, to sort the cell population that specifically express binding partners that bind to the target polypeptide.

Examples of donor-acceptor FRET pairs include FITC and Alexa Fluor 594, although other fluorescence pairs may be used.

In a preferred embodiment, the invention provides a method of identifying a cell which produces a specific binding partner which binds to a target polypeptide, the method comprising the steps:
(a) expressing a library of binding partners in a population of mammalian cells,
wherein each binding partner comprises a framework and a plurality of variable regions, each plurality of variable regions endowing that binding partner with a specific binding affinity for a target,
wherein each cell in the population of mammalian cells expresses at least one member of the binding partner library from one or more retroviral (e.g. lentiviral) vectors which have been integrated into the genome of that cell,
wherein each binding partner comprises a first detectable tag,
wherein each binding partner is secreted from the cell in which it is produced, and
wherein each cell in the population of mammalian cells comprises an expression construct comprising a promoter operably linked to a nucleotide sequence encoding the target polypeptide, wherein the target polypeptide optionally comprises a second detectable tag, wherein the expression construct is integrated into the genome of each cell;
(b) optionally removing cells from the population of mammalian cells to which binding partners bind;
(c) expressing the target polypeptide from the expression construct such that the target polypeptide is displayed on the outer surface of each cell in the population of mammalian cells; and
(d) isolating or identifying cells within the population of mammalian cells to which specific binding partners are bound,
wherein cells to which specific binding partners are bound are isolated or identified using:
   (i) flow cytometry, or
   (ii) magnetic sorting, or
   (iii) a micro-fluidics system wherein cells from the population of mammalian cells are contained within each chamber in a plurality of isolated chambers,
      wherein the cells to which specific binding partners are bound are ones which produce specific binding partners which bind to the target polypeptide,
      wherein each binding partner comprises a first detectable tag (preferably HA), wherein the target polypeptide comprises a second detectable tag (preferably Myc epitope), and wherein the first and second detectable tags are detected using independent antibodies to which are attached first and second members of a donor-acceptor FRET pair (preferably FITC and Alexa Fluor 594).

In a preferred embodiment, the binding partner is an HA-tagged scFV linked to FITC. This is preferably used with a second antibody to a Myc tag on the target polypeptide linked to Alexa Fluor 594.

Bringing these two fluorophores into proximity by binding of the second antibody to the target polypeptide and the scFv binding to the target polypeptide would allow excitation at one wavelength of FITC and detection of an emission at a second wavelength by the Alexa Fluor 594.

One consequence of this detection method may be that only specific binding partner/target polypeptide interactions are detected since FRET is highly sensitive to the distance between donor and acceptor dipoles within the 1-10 nm range.

FRET may be used in any of the isolation/identification systems in Step (d).

The method of the invention may be carried out in a liquid medium, in a semi-solid medium, in a solid medium (e.g. gel) or the cells may be fully or partially immobilised.

Preferably, the method is carried out in a liquid medium, e.g. in an aqueous physiological medium, for example an aqueous physiological medium which is suitable for cell culture and for binding of potential binding partners to the target polypeptide. In this embodiment, the secreted binding partners will be in solution and hence they will be free to bind not only to the cells from which they were secreted, but also to other (e.g. neighbouring) cells. In such a case, the first cells within the population of cells to which specific binding partners are bound will be ones which produce that specific binding partner. Over time, the specific binding partners will be capable of contacting cells (e.g. by convecting or diffusing to them) other than those from which they were secreted, but in reasonably static systems the specific binding partners will bind first to cells from which they were secreted (if the specific binding partners are capable of binding to the target polypeptide).

If the method of the invention is carried out in liquid media, therefore, it may be necessary to assay the cells for any binding of specific binding partners at a number of different time points (e.g. 4 hours, 6 hours, 24 hours, 48 hours, etc.) in order to establish when the cells first become bound by internally-produced specific binding partners. Such cells may then be sorted or isolated (e.g. by fluorescent activated cell sorting (FACS) in the case of fluorescently-labelled binding partners).

In some embodiments, therefore, Step (d) comprises the step:
(d) identifying or isolating cells within the population of cells to which the specific binding partners first become bound.

In other embodiments, Step (d) comprises the step:
(d) identifying or isolating cells within the population of cells to which specific binding partners are bound after a time point when the specific binding partners may only be bound to target polypeptides which are displayed on cells from which the specific binding partners themselves have been secreted.

In light of the fact that, in most methods, there will be very few cells which secrete specific binding partners which are capable of binding to the target polypeptide, problems with the convection or diffusion of specific binding partners to other cells are not considered to be significant.

A further way to reduce the effects of such diffusion is to replace the liquid medium in a continuous or discontinuous manner, thus removing any diffusing specific binding partners from the liquid medium.

In other embodiments, therefore, Step (a) additionally comprises the feature: wherein the method is carried out in a liquid medium which is replaced in a continuous or discontinuous manner.

In some embodiments of the invention, it is desirable to inhibit the movement of the specific binding partners away from the cells in which they are produced. This helps to reduce the level of non-specific background binding and to avoid the production of false-positive cells.

One way to do this is to perform the method of the invention using a liquid medium whose dynamic viscosity is greater than that of water at 25° C. In this way, diffusion of binding partners away from the cells in which they are produced is reduced. The dynamic viscosity of water is 8.9×10⁻⁴ Pa·s at 25° C. Preferably, the dynamic viscosity of the liquid medium in which Step (a) of the method of the invention is carried out is at least 10×10⁻⁴ Pa·s at 25° C.

More preferably, the dynamic viscosity of the liquid medium in which Step (a) of the method of the invention is carried out is between 1×10⁻⁴ Pa·s and 10 Pa·s at 25° C., even more preferably between 0.01 Pa·s and 1 Pa·s at 25° C., and most preferably between 0.01 Pa·s and 0.1 Pa·s at 25° C.

For example, the dynamic viscosity of the liquid medium may be increased using a neutral viscosity increaser, e.g. a sugar, polyvinylpyrrolidine (PVP), polyethylene glycol (PEG, molecular weight up to 20 KDa, more preferably about 8 KDa, up to 50% vol/vol) or poly[N(2-hydroxypropyl)methacrylamide] (preferably 10-100 KDa, up to 40% wt/vol). A further way to prevent diffusion of the binding partners away from the cells in which they are produced is to carry out the method of the invention in a gel. A gel is a solid jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinking within the fluid that gives a gel its structure (hardness) and contributes to the adhesive stick (tack). In this way, gels are a dispersion of molecules of a liquid within a solid in which the solid is the continuous phase and the liquid is the discontinuous phase.

Preferably, the gel is a hydrogel, i.e. a cross-linked network of hydrophilic polymer chains. In some embodiments, the hydrogel is formed from polyvinyl alcohol, sodium polyacrylate, acrylate polymers, or polymers or copolymers with an abundance of hydrophilic groups such as copolymers based on poly[N(2-hydroxypropyl) methacrylamide] or block copolymers based on polyethylene glycol or oligopeptides. In other embodiments, the hydrogel is formed from alginate, agarose, methylcellulose, hyaluronan, or other naturally-derived polymers.

Preferably, the gel is an alginate hydrogel, more preferably a calcium alginate hydrogel. Cells which are entrapped within such a gel may readily be released upon application of a divalent cation chelator, e.g. EDTA or EGTA, and then the ‘labelled’ cells may be isolated (e.g. by flow sorting) in the normal way. The hydrogel may be in the form of beads.

The background level of non-specific binding of the specific binding partners to the cells can be reduced by a negative selection step. In this step, cells to which binding partners are (non-specifically) bound before the target polypeptide is displayed on the cells (i.e. before induction of expression of the target polypeptide in embodiments wherein the expression construct comprises an inducible promoter) are first removed from the cell population.

In some embodiments, therefore, Step (a) additionally comprises the step:
removing cells from the population of cells to which binding partners bind.

Once the cells which produce specific binding partners against the target polypeptides have been identified and/or isolated (i.e. those to which the specific binding partners bind), those cells may be purified, by any suitable means.

In some embodiments, the cells to which the specific binding partners bind are purified more than once (i.e. reiteratively), e.g. by flow cytometry.

The polynucleotides which encode the specific binding partners which are produced by the purified cells may be sequenced, thus providing the amino acid sequence of all or part of the specific binding partners. Preferably, one of more of the regions of the specific binding partners which are not common between the members of the binding partner library are sequenced. More preferably, the amino acid sequences of one or more of the specific binding partners' CDR sequences are obtained.

Preferably, the sequencing method is a non-Sanger sequencing method. Such methods include sequencing by synthesis (e.g. Illumina, Inc.), nanopore sequencing (e.g. Oxford Nanopore Technologies), pac-bio sequencing (e.g. Pacific Biosciences) and other next generation sequencing (NGS) methods.

The amino acid sequences of the most promising specific binding partners may then be subjected to affinity maturation, e.g. by mutagenesis of the nucleotide or amino acid sequence, in order to produce specific binding partners with higher affinity or specificity for the target polypeptide.

In a further embodiment, the invention provides a specific binding partner which has been identified by a method of the invention.

In yet a further embodiment, the invention provides a process for producing a population of mammalian cells, the process comprising the steps:
(A) integrating, into the genome of each (or substantially each) cell in the population of mammalian cells, an expression construct comprising a promoter (preferably an inducible promoter) operably linked to a nucleotide sequence encoding a target polypeptide; and
(B) contacting the population of mammalian cells with a library of retroviral (e.g. lentiviral) particles, each particle comprising a retroviral (e.g. lentiviral) vector encoding a member of a binding partner library and a detectable tag;
   so as to produce a population of mammalian cells wherein each cell (or substantially each cell) in the population of mammalian cells secretes one or more binding partners (e.g. antibodies or antibody mimetics), and
   wherein each cell in the population of mammalian cells displays or is capable of displaying (preferably upon induction) the target polypeptide on the outer surface of the cell.

Steps (A) and (B) may be carried out in either order.

Preferably, each cell (or substantially each cell) in the population of cells secretes or is capable of secreting 1-3, 1-2 or, most preferably, just 1 binding partner.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. The disclosure of each reference set forth herein is incorporated herein by reference in its entirety.

### Example 1: Demonstration of Self-Labelling by Cells Expressing Anti-EpCAM Antibodies

Epithelial cell adhesion molecule (EpCAM) was selected as a suitable target polypeptide (bait antigen). EpCAM is a glycosylated, 30- to 40-kDa type I membrane protein containing three potential N-linked glycosylation sites.

HEK293 cells were transfected with an EpCAM-expression construct (HEK293 cells are normally EpCAM negative) together with a secreted HA-tagged anti-EpCAM single chain antibody expression-construct. After a 24-hour incubation period, the cells were stained with a fluorescently-labelled anti HA-tag antibody and analysed by flow cytometry to determine which cells had self-labelled their membrane EpCAM with the encoded anti-EpCAM antibody.

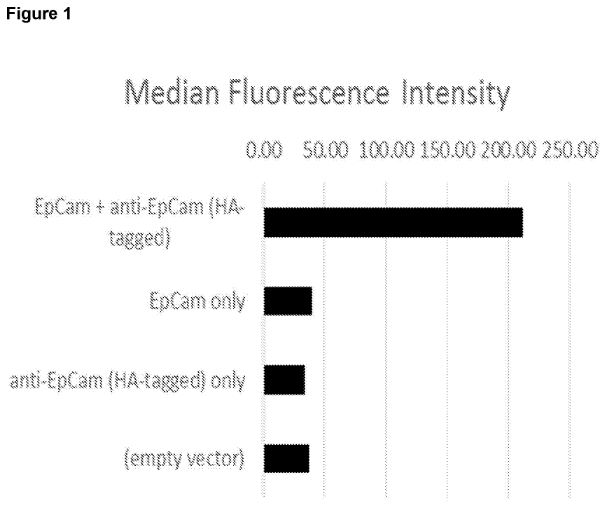

The results are shown in FIG. 1. Cells expressing both the ‘bait’ antigen (EpCAM) and the scFv became highly fluorescent, whereas all of the other cells (expressing either EpCAM only or the scFv only) did not. This shows that cells secreting scFv can label antigens on their own surface.

### Example 2: Optimising Stringency to Prevent Labelling of Irrelevant Cells

Two populations of EpCAM-expressing cells were made. Some expressed the anti-EpCAM antibodies, while others instead expressed green fluorescence protein (GFP). Cells were mixed at different ratios (always with the antibody-expressing cells in the minority), incubated for different times, fixed and surface-bound antibody was visualised by staining in the red channel. The small number of cells expressing the antibody invariably labelled themselves first, giving rise to cells in the lower right hand quadrant of the flow cytometry plot (cells were red but not green, indicating that the antibody-producing cells were labelled before the irrelevant cells).

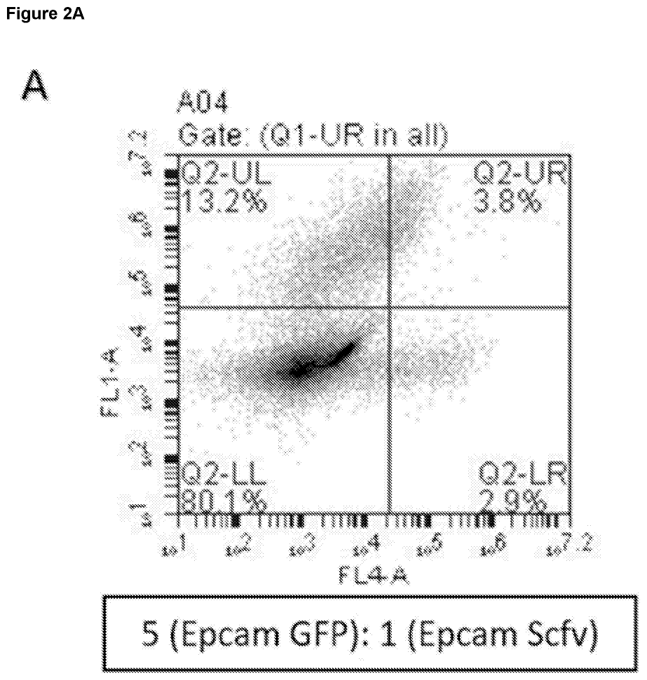

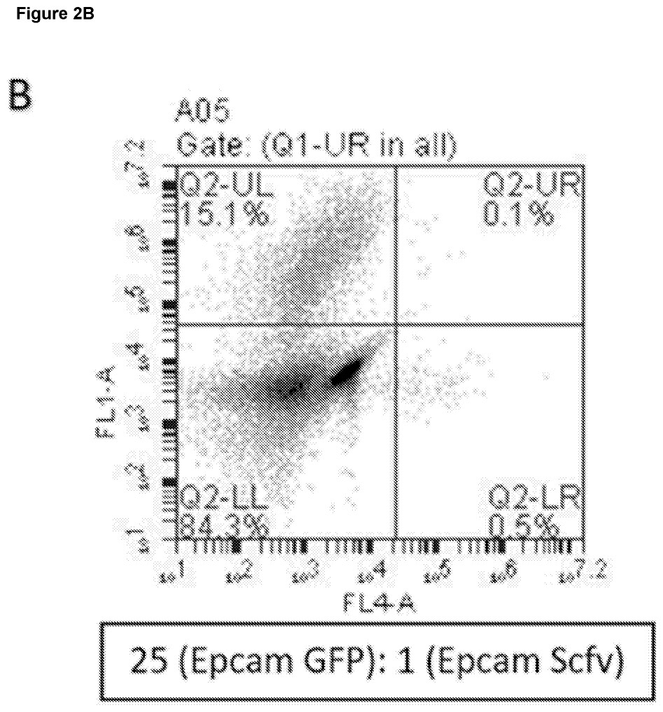

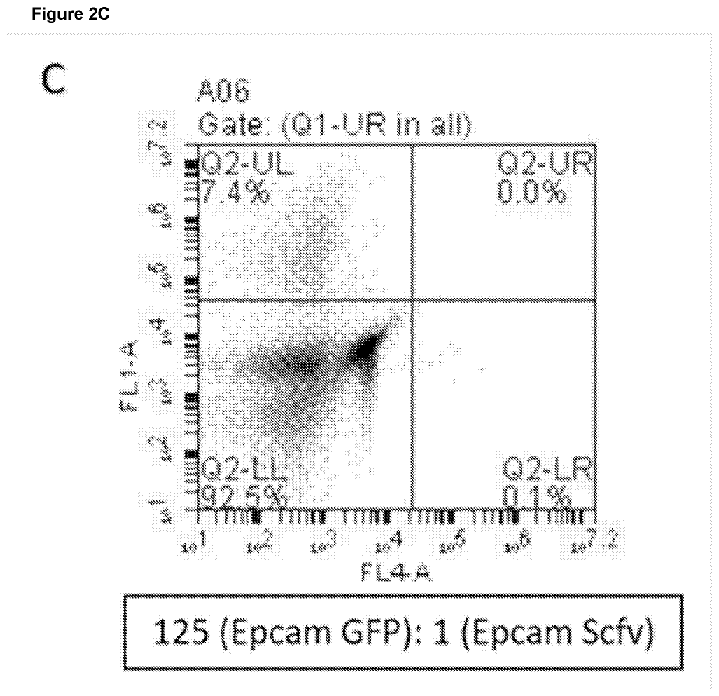

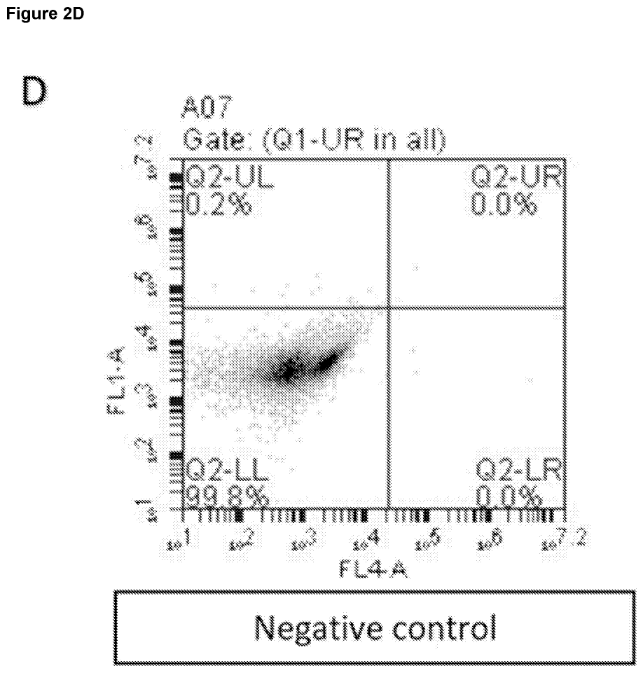

The results are shown in FIG. 2A to 2D. Even at a dilution of 1:125, an appreciable number of cells appear in the lower right quadrant, representing cells expressing antibodies that bind the cell-surface antigen.

### Example 3: Production of Antibodies to DRD1

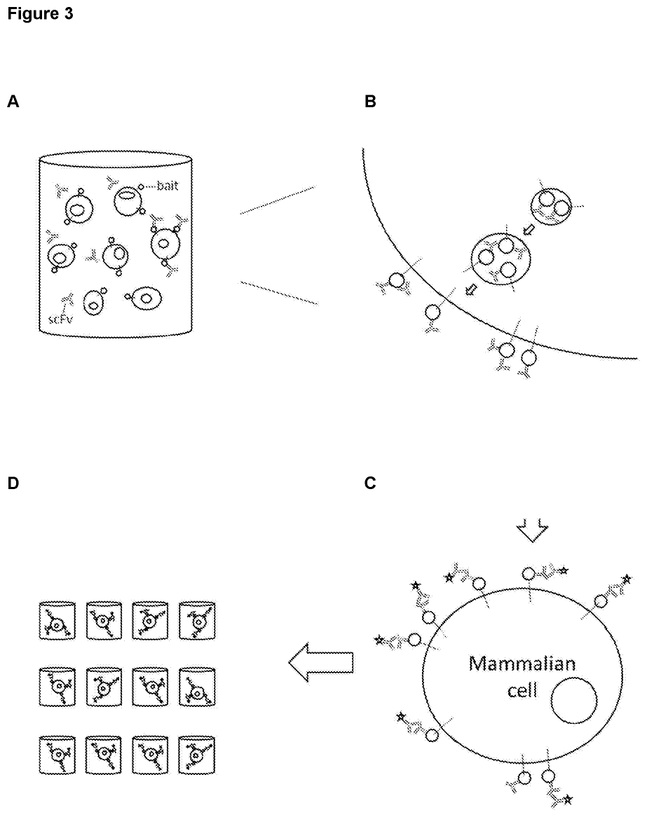

In this example, the target polypeptide (bait) is DRD1; this is expressed in CHO cells (see FIG. 3 for an overview). A retrovirus transfer vector is used to clone the target polypeptide (bait) construct and the antibody libraries into the CHO cells.

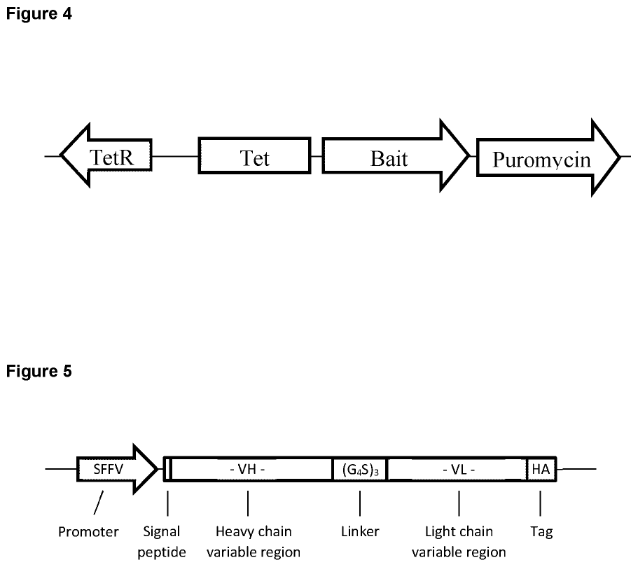

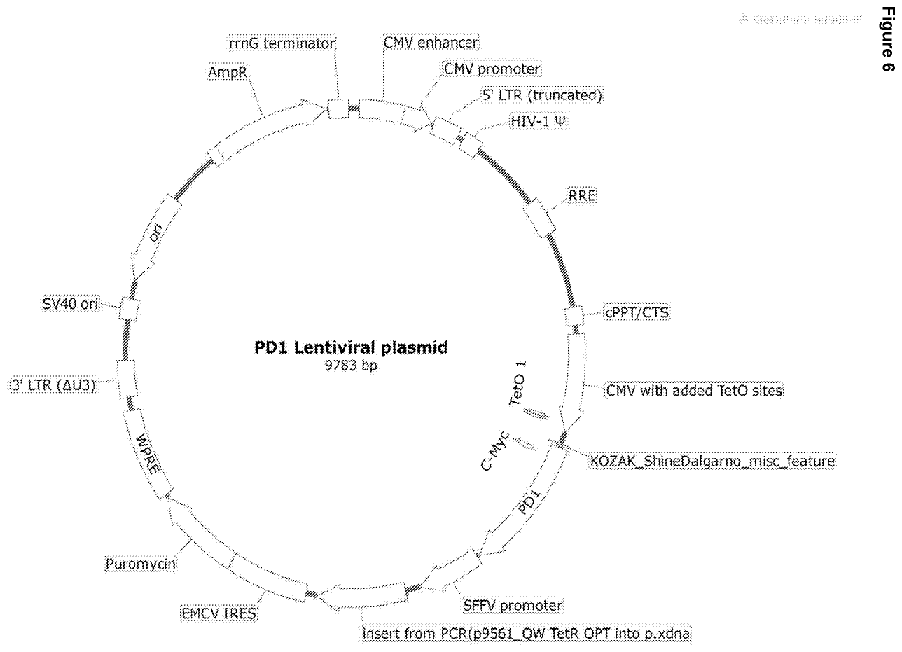

The target polypeptide (bait) cell lines are produced by using the retrovirus system to integrate the gene for the target polypeptide (bait) into the host cell (CHO) genome along with a selectable marker (see FIG. 4). The target polypeptide construct also contains the gene for the Tet repressor (TetR). Target polypeptide (bait) expression is driven by a doxycycline-inducible promoter.

A library of retrovirus particles encoding a cDNA-based library of human scFv sequences of human-like scFv sequences is used to infect the CHO cells. For the scFv libraries, the retrovirus transfer vector is modified to contain a constitutive promoter (SFFV) and the flanking regions of the scFv antibody subunits (see FIG. 5). Each scFV also includes a HA tag.

After a 24-hour incubation period, the cells are stained with a fluorescently-labelled anti HA-tag antibody and analysed by flow cytometry to determine which cells had self-labelled their membrane DRD1 with a scFV antibody.

### Example 4: Selection of a PD-1 Binder Using MACS Technology

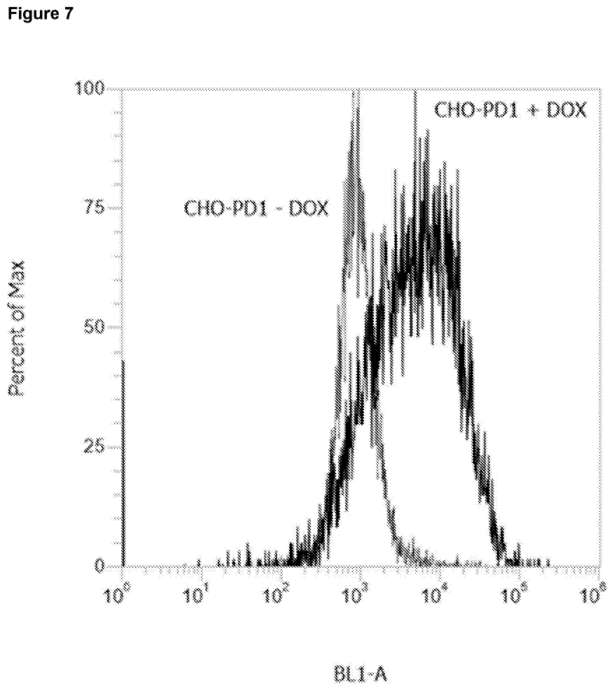

A lentivirus vector was constructed that contained the gene for human programmed cell death protein 1 (PD-1, SEQ ID NO: 1) with an N-terminal Myc tag (EQKLISEEDL, SEQ ID NO: 2) under the control of a doxycycline inducible promoter. The vector also included a puromycin selectable marker and the expression cassettes were flanked by lentiviral long terminal repeat (LTR) sequences (FIG. 6). This plasmid was co-transfected into a HEK293 cell line together with plasmids encoding the necessary genes for virus particle assembly supplied in trans (for overview of methodologies see Sakuma et al. 2012. Lentiviral vectors: basic to translational. Biochem J. 443(3):603-618). The secreted virus particles containing the packaged PD-1 gene were harvested and used to transduce CHO cells at an MOI of 0.5. Transduced cells were selected with puromycin and grown out as a cell pool. The selected cells were grown for 4 days in the presence of doxycycline and PD-1 expression was confirmed by flow cytometry (FIG. 7).

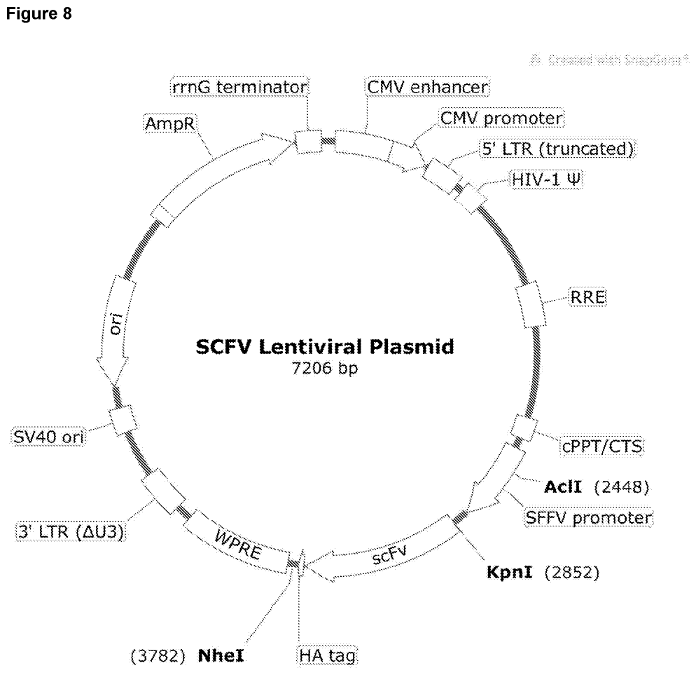

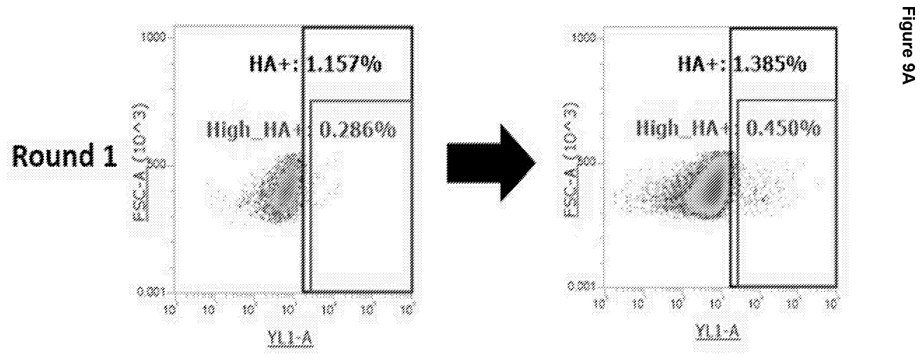

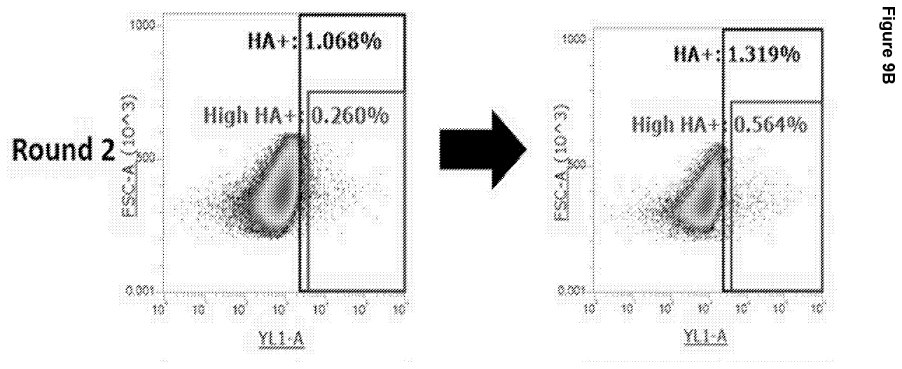

A library of DNA fragments encoding a diverse repertoire of scFv protein sequences (SEQ ID NO: 3) was generated by gene synthesis (Oak Biosciences, Sunnyvale, Calif.) that contained single antibody VL and VH genes separated by an 18 amino acid linker (GGSSRSSEVQLVESGGG, SEQ ID NO: 4) with amino acid variations within CDRH3 and CDRL3 to give a total theoretical library diversity of 1×10⁹ library members. These fragments were cloned into a lentiviral vector under the control of a spleen focus-forming virus (SFFV) promoter and included an upstream signal sequence (immunoglobulin light chain signal sequence) to direct secretion of the scFv into the growth medium and a downstream HA tag for detection. The expression cassette was flanked by lentiviral LTRs (FIG. 8). A library of virus particles was generated as described above.

A further lentiviral vector was constructed, similar to the scFv library vector but containing a single scFv sequence based upon the variable domain sequences of an anti-PD-1 antibody (pembrolizumab, WO2012135408, SEQ ID NO: 5) separated by a 15 amino acid (G₄S)₃ amino acid linker (SEQ ID NO: 6).

In order to demonstrate the principle of the antibody discovery process, 10⁹ PD-1 transduced CHO cells grown in the presence of doxycycline for 24 h were further transduced at an MOI of 1 with a mixture of the scFv library containing lentiviruses with anti-PD-1 containing lentiviruses at a ratio of 106:1. The transduced cells were grown for 28 h at 37° C. and 16 h at 25° C. before cells were harvested by centrifugation. Cells were resuspended in 180 mL of PBS pH 7.4 containing 10% FBS and 2 mM EDTA (“MACS buffer”) and passed through a 40 μm cell strainer. 2×10⁹ cells were centrifuged and resuspended in 20 mL of MACS buffer at a density of 1×10⁸ cells/mL and 2 mL of anti-HA antibody conjugated with phycoerythrin (PE) (Miltenyi Biotech, Bisley, UK). Cells were incubated at 4° C. for 15 minutes and washed twice in MACS buffer. For magnetic labelling cells were resuspended in 16 mL of MACS buffer and incubated with 4 mL of anti-PE MicroBeads (Miltenyi Biotech, Bisley, UK) at 4° C. for 15 minutes. Cells were washed again and resuspended in 10 mL of MACS buffer before being passed through a magnetic separation column (Miltenyi Biotech, Bisley, UK) by gravity flow. The columns were washed twice with MACS buffer before bound cells were eluted with MACS buffer. Magnetic enrichment of the labelled cells was repeated over a second MACS Column.

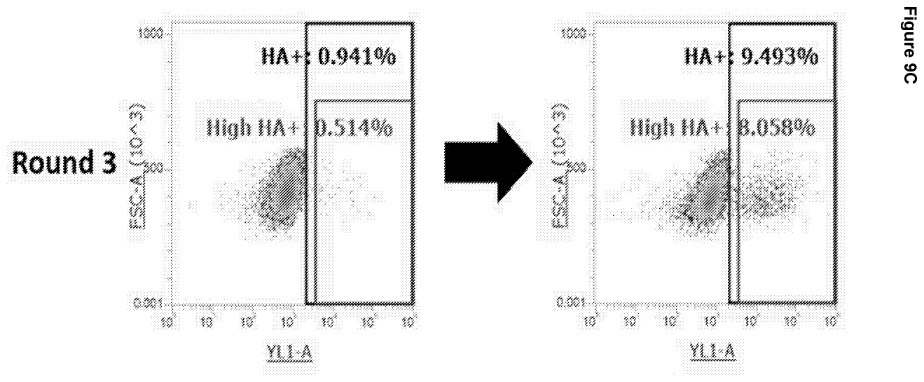

Samples of labelled cells obtained before and after the positive magnetic selection were analysed for PE fluorescence on the Attunem NxT Acoustic Focusing Cytometer (ThermoFisher Scientific, Loughborough, UK). Eluted cells were returned to growth medium and expanded for at least 4 days before starting a new round of positive selection. Samples (5×10⁸ cells) were subjected to two further rounds of positive magnetic selection as described above, including doxycycline induction 3 days before MACS as well as analysis after each stage by flow cytometry (FIGS. 9A to 9C). After the third round of positive selection, a negative selection was performed to remove cells secreting scFv from the library that bound either non-specifically to the CHO cells or bound to proteins other than human PD-1 on the cell surface: in this case cells were cultured for 8 days without doxycycline in the growth medium to turn off the expression of PD-1, before repeating the selection process as described above with 5×10⁷ cells applied to the column and collecting the flow-through and the first wash fraction rather than the eluate.

For the purposes of this Example, the negative selection step was performed after the third round of positive selection; however, this step could be performed at any stage in the selection process: before performing positive selection, or after the first, second or third rounds of positive selection. Furthermore, any number of positive selections and/or negative selections may be done in any combination as necessary for the isolation of specific binders against the target protein.

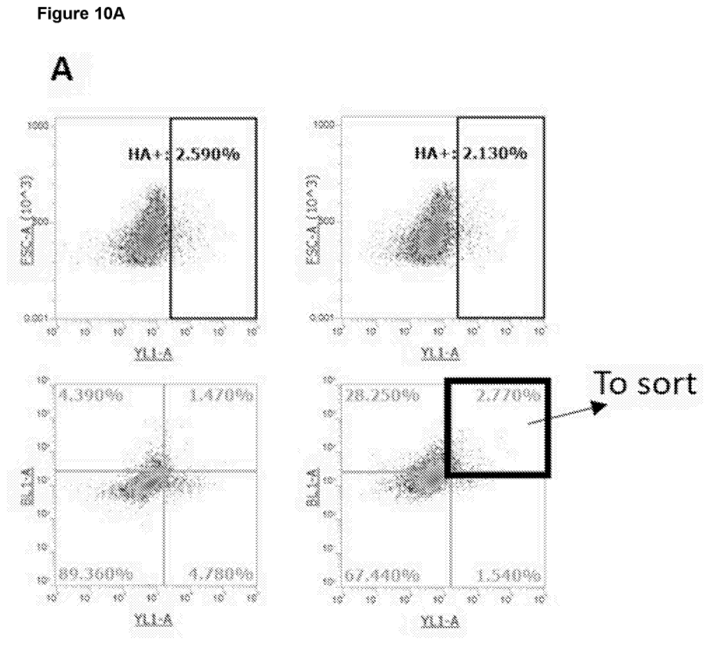

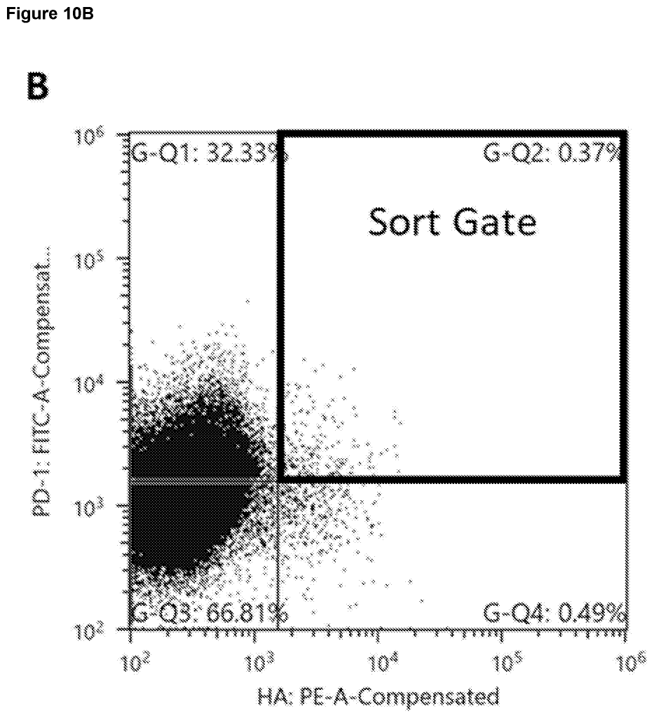

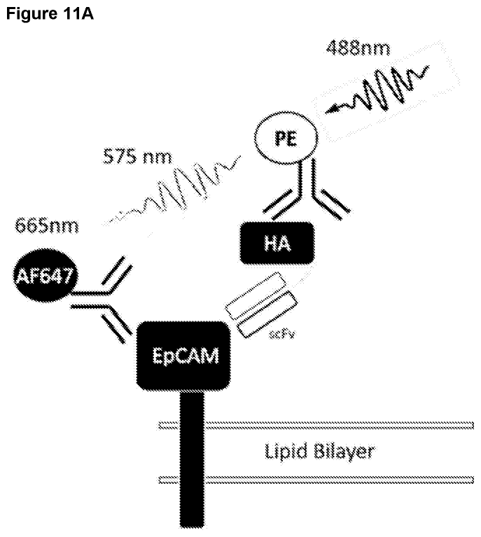

Following negative selection, the recovered fraction was expanded for 3 days (48 h at 37° C. followed by 18 h at 25° C.) in the presence of doxycycline to turn the PD-1 expression back on before sorting cells using FACS (SH800 Cell Sorter, Sony Biotechnology, Weybridge, UK). For this purpose, cells were double stained with anti-HA-PE (Miltenyi Biotech, Bisley, UK) and anti-Myc-FITC (Abcam, Cambridge, UK) antibodies. SYTOX™ AADvanced™ Dead Cell Stain Kit (ThermoFisher Scientific, Loughborough, UK) was used as a viability dye to exclude dead cells. Appropriate single-stained compensation controls were used to set up automatic compensation using the SH800 software. Double stained cells (FIGS. 10A and 10B) were collected, lysed and genomic DNA purified using a DNeasy Blood & Tissue Kit (Qiagen, Manchester, UK) according to the manufacturer's instructions.

Genomic DNA from the sorted cells was amplified by PCR using primers that flank the scFv expression cassette (SEQ ID NO: 7 and SEQ ID NO: 8) and the amplicons were sequenced using paired end MiSeq next generation sequencing (NGS) technology (Illumina, Cambridge, UK). The sequencing data was analysed to identify the CDRs of the enriched scFvs and showed that only a limited number of scFv had come through the selection process, of which 18% were the sequence of pembrolizumab, thus demonstrating an approximate 200,000 enrichment of specific PD-1 binders using the methodology described above.

### Example 5: Isolation of CHO Cells Secreting Antigen-Binding scFv Using Microfluidics

PD-1 transduced CHO cells, produced as described in Example 4, are expanded in the absence of doxycycline and further transduced with lentiviruses containing a library of scFv also as described in Example 4. The transduced cells are grown for 28 h at 37° C. and 16 h at 25° C. in the absence of doxycycline before being harvested by centrifugation. The cells are prepared for MACS separation and passed down a MACS column as described in Example 4 with the flow-through and first wash fraction being collected. These fractions are pooled, centrifuged and the cells resuspended in MACS buffer and applied to a second MACS column where the flow-through and first wash fraction are again collected. This represents a first negative selection step. The recovered cells are returned to growth medium containing doxycycline and expanded for at least 4 days before performing a round of positive selection as described in Example 4.

Cells following the positive selection step are expanded in growth medium containing doxycycline for 4 days before being centrifuged, washed and resuspended in encapsulation medium (growth medium containing 16% OptiPrep™ (Sigma-Aldrich, Poole, UK), 0.1% Pluronic® (ThermoFisher Scientific, Loughborough, UK), anti-HA tag FITC (1:200, Miltenyi Biotech, Bisley, UK) and anti-Myc tag Alexa Fluor 594 (1:200, Abcam, Cambridge, UK)) at a density of 1×10⁸ cells/mL. The cell suspension is placed in the reservoir of a Cyto-Cartridge® (Sphere Fluidics, Cambridge, UK) and the cartridge loaded into a Cyto-Mine® instrument (Sphere Fluidics, Cambridge, UK). Droplets each containing an average of 30 CHO cells in 200 pL of medium are encapsulated in the Cyto-Surf® reagent (Sphere Fluidics, Cambridge, UK) and directed to the cartridge incubation chamber where they are incubated at 37° C. for 2 to 4 hours to promote scFv expression. The droplets are then passed through the detection module of the cartridge which can analyse both green fluorescence and red fluorescence and discriminate between diffuse label in the medium and label concentrated around the cell surface. Droplets containing cells that are labelled at the cell surface with both anti-Myc Alexa Fluor 594 (indicating the presence of PD-1 on the cell surface) and anti-HA FITC (indicating bound scFv) are directed to a collection chamber in the cartridge and then distributed into 384 well plates pre-filled with 50 μl per well of growth medium containing doxycycline at one droplet per well.

The plates are incubated at 37° C. for 48 hours and the cells recovered from each well and combined into a cell pool. The cell pool is then expanded for a further 48 hours before being centrifuged and washed and resuspended in encapsulation medium at a density of 1.2×10⁶ cells/ml. The Cyto-Mine® run is then repeated as above except that the cells are encapsulated at a maximum density of one cell per droplet. The droplets are passed through the cartridge as described above, detecting on the red and green fluorescence channels. Additionally, the droplets are analysed by Förster resonance energy transfer (FRET) by excitation of the FITC fluorophore and analysing for emission by the Alexa Fluor 594 fluorophore. Droplets that are positive for both green and red emission indicate the presence of cells that are positive for PD-1 expression that are also labelled with scFv. Droplets that additionally exhibit a FRET signal indicate that the FITC and Alexa Fluor 594 labels are in close proximity (<10 nM apart) and the signal is therefore more likely to be as a result of a specific interaction between the scFv and PD-1 rather than a non-specific interaction between the scFv and other proteins on the cell surface. Selected droplets are directed to a collection chamber in the cartridge and then distributed into 384 well plates pre-filled with 50 μl per well of growth medium at one droplet per well.

If required, the cells are further analysed for the presence of non-specific binders by growing in medium containing doxycycline before centrifuging and washing and resuspending at a cell density of 1.2×10⁶ cells/ml in encapsulation medium also containing 1.2×10⁷ of parental CHO cells (i.e. cells that do not express PD-1). This cell suspension is loaded into the Cyto-Mine® and encapsulated into droplets each containing an average of 10 cells. The droplets are processed through the instrument as described above analysing for red and green fluorescence. Droplets that exhibit a low level of red fluorescence (PD-1 expression on one or two cells) and high green fluorescence indicate the presence of scFv that are binding to all the cells in the droplet and are therefore binding non-specifically to CHO cells. Droplets that exhibit no fluorescence do not contain a cell expressing scFv. Droplets that exhibit positive but low red fluorescence (PD-1 expression on one or two cells) and positive but low green fluorescence (scFv staining of one or two cells) indicate the presence of scFv binders that are specific for PD-1. These droplets are directed to a collection chamber in the cartridge and then distributed into 384 well plates pre-filled with 50 μl per well of growth medium containing doxycycline at one droplet per well. The collected cells are then pooled, expanded in growth medium containing doxycycline and processed through the Cyto-Mine® at a maximum of 1 cell per droplet, as described above, in order to re-isolate the single cells expressing scFv that are specific for PD-1 that are maintained in clonal cultures derived from the single sorted cells without pooling.

Samples of cells from each culture are lysed and the genomic DNA isolated and sequenced by Sanger Sequencing to identify the DNA and protein sequences of the positive scFv. Heavy and light chain variable region pairs are cloned into expression vectors for the expression of whole antibodies, which are then transiently transfected into HEK 293 cells. Cell culture supernatants are assayed for the presence and quantity of expressed antibody by ELISA and then analysed by flow cytometry for binding to CHO cells expressing PD-1.

### Example 6: Use of FRET to Detect scFv Binding to EpCAM

In order to increase the specificity of the antigen-binding signal by secreted scFvs, a Fluorescence Resonance Energy Transfer (FRET) signal between an acceptor and donor fluorophore which co-bound the target-scFv complex was used. The membrane associated target antigen EpCAM was bound by a FRET acceptor antibody which bound to a Myc affinity tag fused to the antigen. The FRET donor antibody bound to a HA tag on the scFv. If the secreted scFv bound to the cell surface antigen, then the acceptor/donor were brought within the Förster distance and generated a FRET emission from the acceptor fluorophore, once the donor had been excited. This signal was then utilised in a FACS experiment to sort the cell population that specifically expressed scFvs that bound to the cell surface target antigen.

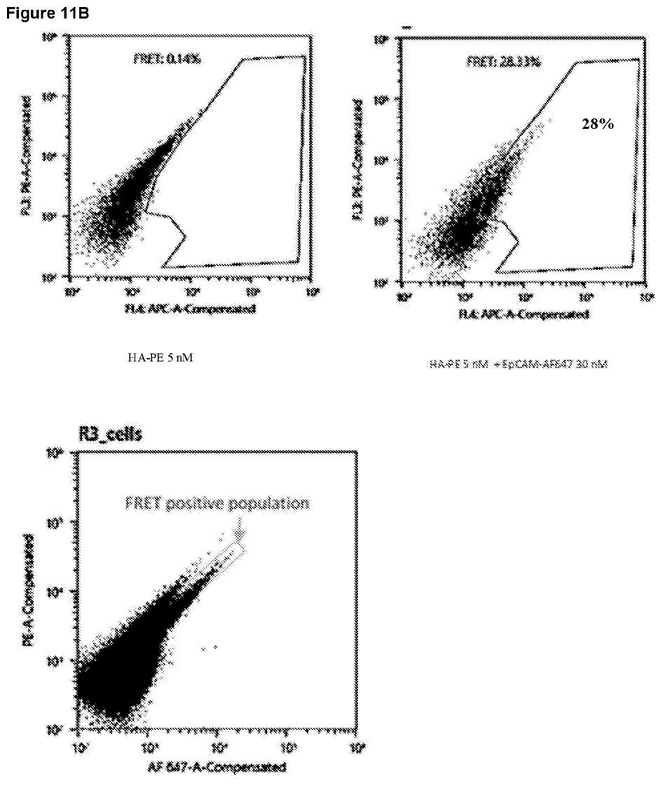

The experimental set-up and results are shown in FIGS. 11A and 11B. This figure shows a schematic diagram of scFv binding to EpCAM generating FRET by co-binding of anti-HA-PE and anti-MYC-AF647 antibodies to the cell surface complex (left panel) (FIG. 11A). FACS data of CHO cells co-expressing EpCAM and an scFv which bound EpCAM is shown in the right panel (FIG. 11B). The left panel shows single staining with anti-HA-PE: the right panel shows dual staining with donor and acceptor fluorophores (PE and AF647). The gated fraction (lower panel) highlights the cell population that exhibited FRET.

### Example 7: Use of “Capturing Cells” to Reduce Background Signal

During the screening and selection of the mammalian cells, soluble antibodies are continually released by the transduced cell subpopulation. Undesirably, these antibodies can bind to their epitopes (either on the desired target or any membrane protein) on neighbouring cells, leading to the contamination of the sorted population with the cross-labelled cells.

In order to reduce this cross-labelling, a CHO cell sample of interest was co-cultured with a 1:4 or 1:9 excess of non-transduced CHO cells (“capturing cells”) expressing a PD1 target antigen. These capturing cells sequestered the scFvs antibodies which were secreted into the media. Prior to co-culture, the capturing cells were labelled with a fluorescent intracellular dye (CellTracker™ Blue CMAC, Thermofisher Scientific, Loughborough, UK). The mixed cell samples were incubated for 2-4 days in the presence of doxycycline in order to induce target antigen expression on each cell. Cells were harvested and stained with a suitable antibody pair (anti-Myc-FITC and anti-HA-PE). Since the emission profile of the intracellular dye did not overlap with any of the fluorescently-labelled antibodies, the level of CMAC fluorescence could be used to distinguish the capturing cells from the cell sample, and to gate out the non-capturing CHO cells for further analysis or sorting

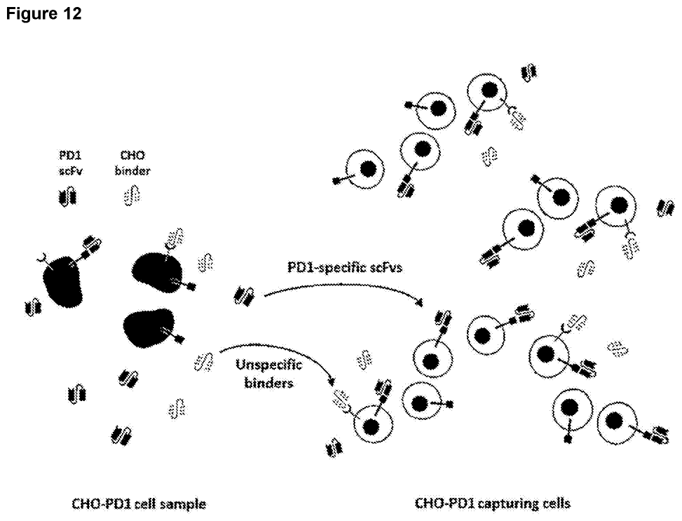

FIG. 12 shows a schematic diagram of the experiment where scFvs which were secreted from the transduced CHO cells were sequestered by the capturing CHO cells labelled with an intracellular dye. This set-up significantly reduced the impact of antibody cross-labelling between cells and allowed for specific isolation of self-labelled cells from culture.

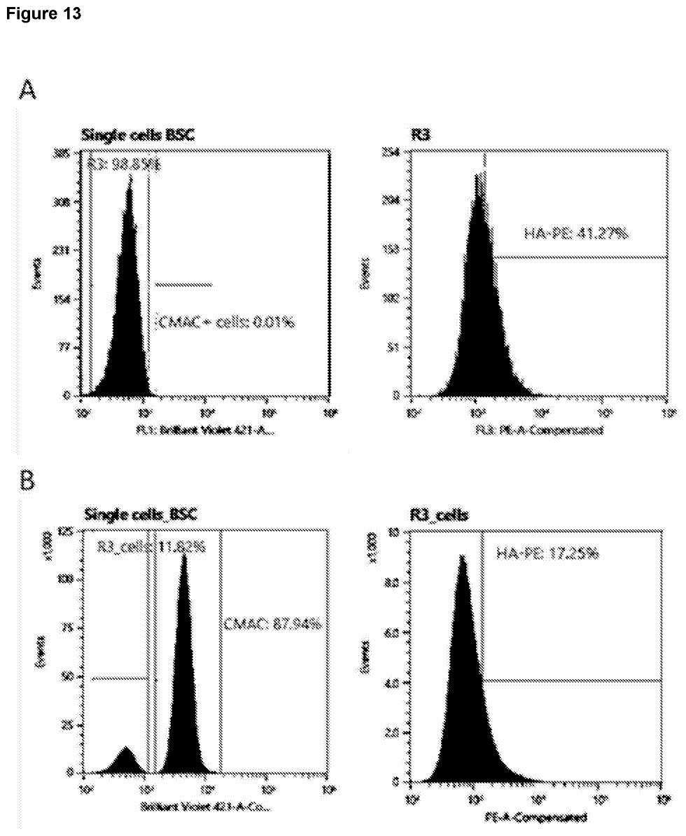

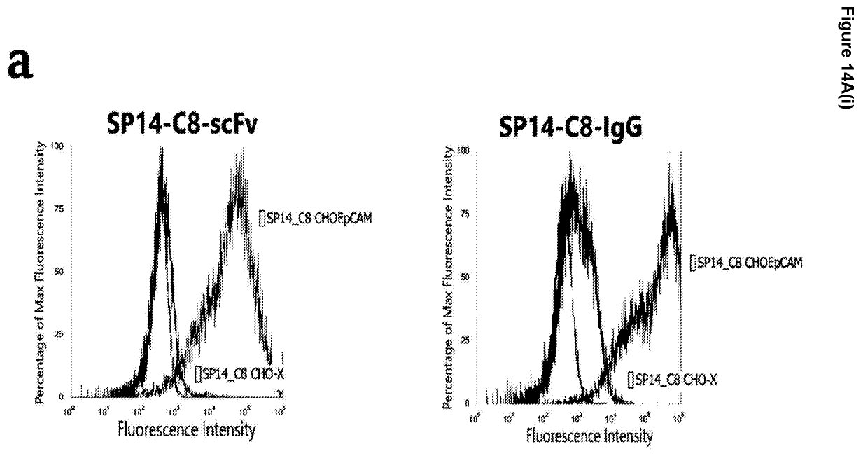

FIG. 13 shows the FACS analysis of the transduced CHO cells which co-expressed a target antigen (PD1) and secreted scFv which bound to the same antigen. The signal was generated from anti-HA-PE monoclonal antibody. The top panel shows anti-HA labelling of the standard transduced cell population (no capturing cells). The lower panel shows the impact of co-culturing with CHO cells expressing the target antigen labelled with intracellular dye (CMAC). The CMAC signal was used to sort away the non-transduced population, thus enabling a reduction in the cross-labelling and FACS sorting of the true self-labelled positive fraction.

### Example 8: Identification of Candidate scFv Sequences, IgG Re-Formatting and Validation Against CHO-EpCAM Cells and SPR

From the output of a second round of FACS enrichment, the variable domain genes from a number of selected EpCAM positive binders were PCR amplified and sequenced. Three clones (SP12-E10, SP14-C8 and SP17-F7) shared a common heavy chain sequence but had different light chain sequences.

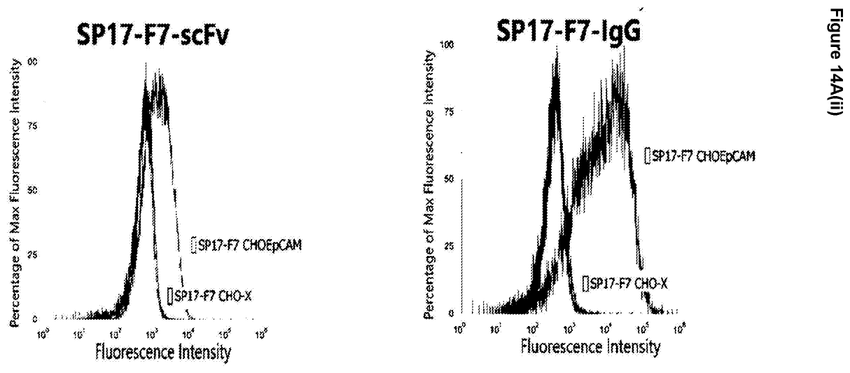

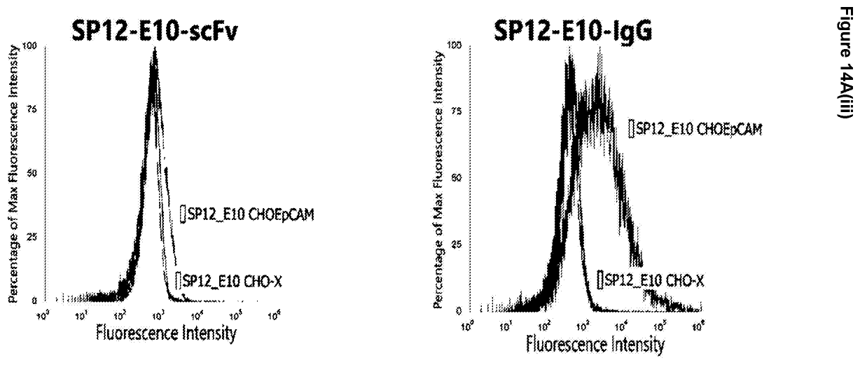

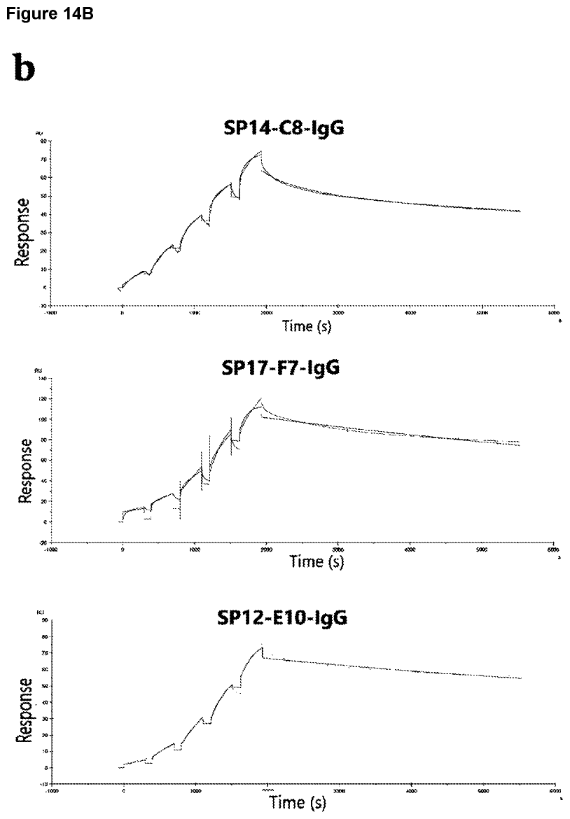

All three sequences were re-cloned as both scFv and full-length IgG1 into expression plasmids and transfected into suspension HEK293 cells modified to express EBNA1 (OX293-EBNA). The supematants from the transfected cells were once again challenged against CHO-EpCAM cells or CHO-X control cells to confirm their specificity. SP12-E10, SP14-C8 and SP17-F7 (FIGS. 14a(i), 14a(ii) and 14a(iii))) were found to bind specifically to CHO-EpCAM cells when formatted as both scFv and whole IgG1. The three positive clones displayed different binding characteristics either as scFv or whole IgG1 to the CHO-EpCAM cells with clone SP14-C8 showing the strongest binding and SP12-E10 the weakest in each case. The IgG1 converted clones were then purified via a protein-A affinity column.

Single-cycle kinetic SPR analysis on a Biacore T200 of SP12-E10, SP14-C8 and SP17-F7 immobilised on a protein A surface using recombinant human extra-cellular domain of EpCAM as analyte gave affinities of 5.92 nM, 0.76 nM and 58.9 nM, respectively (Table 1).

TABLE 1

| Variant | kₐ (1/Ms) | k_{d} (1/s) | K_{D} (M) | EC₅₀ (M) | Chi² (RU²) |
|---|---|---|---|---|---|
| SP14−C8 | 1.28E+05 | 9.65E−05 | 7.57E−10 | 1.09E−08 | 1.55 |
| SP17−F7 | 1.20E+03 | 6.89E−05 | 5.89E−08 | na | 10.9 |
| SP12−E10 | 9.49E+03 | 5.61E−05 | 5.92E−09 | 1.75E−06 | 1.39 |

The association rate constant (Ka) of SP14-C8 (1.28×10⁵ 1/Ms) was significantly faster than for the other two antibodies while the dissociation rate constants (Kd) between all three antibodies varied by less than two-fold. Therefore, using our mammalian display technique, we have isolated an anti-EpCAM specific antibody with an apparent sub-nanomolar affinity.

### Example 9: Generation of CAR-T Constructs with EpCAM scFv and CAR-T Cell Assays

Chimeric antigen receptor (CAR)-T cells were tested with our panel of three EpCAM binders. Two EpCAM positive cell lines, MCF-7 (a breast cancer cell line) and the CHO-EpCAM cell line used for antibody selections, were used as targets and both Jurkat cells and CD3+ T cells derived from human peripheral blood mononuclear cells (PBMC) were used as effector cells. CAR-T cells were generated by lentiviral transduction of either Jurkat cells or CD3+ T cells derived from human PBMC. Lentivirus was engineered to encode a second-generation CAR scaffold with anti-EpCAM scFv fused to a CD8-derived transmembrane region followed by a 4-1BB co-stimulatory domain and a CD3ζ chain.

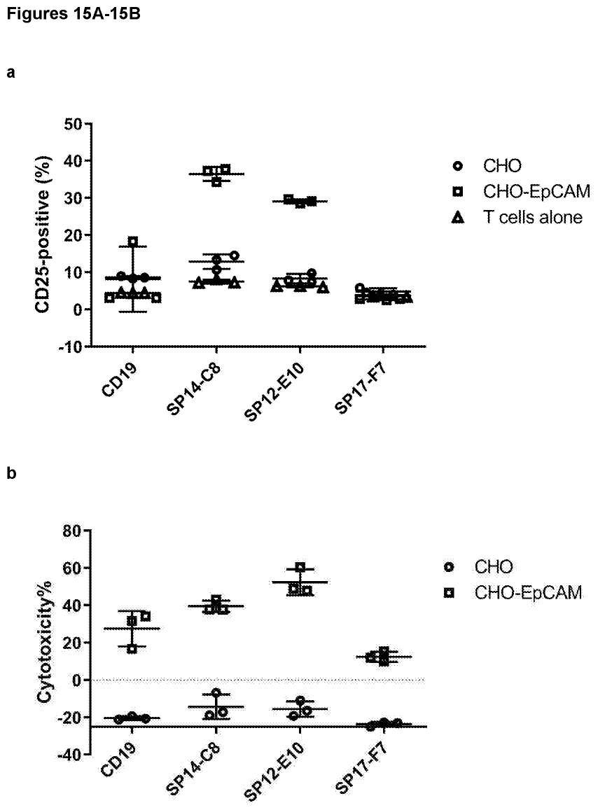

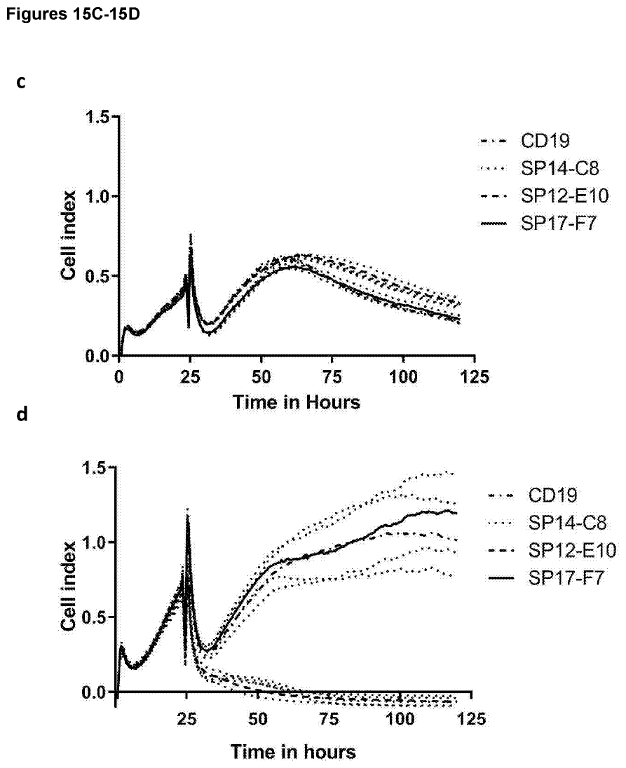

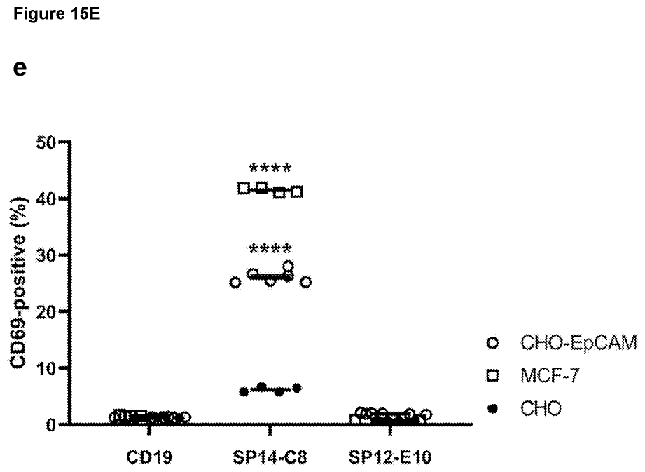

Transduced CD3+ T cells derived from human PBMC were expanded for 10 days before testing. Control CAR-T cells with the same architecture and an scFv recognizing CD19 were also generated. The antigen specificity of the CAR-T cells was tested in a co-culture with CHO-X and CHO-EpCAM cell lines. EpCAM-CAR-T cells displayed a significant increase in the expression of activation marker CD25 (36.5% and 29% by SP14-C8 and SP12-E10, respectively, FIG. 15A) and elicited cell cytotoxicity (40% and 52% by SP14-C8 and SP12-E10, respectively, FIG. 158) only upon co-culture with target CHO-EpCAM cells and not with parental CHO-X cells. The control CD19-CAR-T cells did not display activation since CD25 expression was similar to the background level observed with T cells alone. An increase in the cytotoxicity against CHO-EpCAM cells was however observed (27%, FIG. 15B), suggesting a degree of cross-reactivity. We further explored the CAR-T cell induced target cell killing in co-cultures of CHO-X and CHO-EpCAM cells measured in real-time by cell index, a unitless measure of cell viability. In the presence of control CD19-CAR-T cells, CHO-X and CHO-EpCAM cells persisted for 120 hours (FIG. 15C) whereas with EpCAM-CAR-T cells (SP14-C8 and SP12-E10), complete lysis of CHO-EpCAM cells was observed within 30 hours (FIG. 15D). No cell cytotoxicity was observed either with EpCAM-CAR-T cells (SP14-C8 and SP12-E10) in parental CHO-X cells or with CD19-CAR-T cells with either cell line. **

Finally, the anti-EpCAM scFv CAR constructs were tested against MCF7 and CHO-EpCAM cells using lentivirus transduced Jurkat cells as effectors. CAR transduced Jurkat cells were generated with the same SP14-C8, SP12-E10 and CD19 lentiviral constructs as above to assess activation upon co-culture with EpCAM expressing target cells. CAR Jurkat cells were incubated with parental CHO—X, CHO-EpCAM and MCF7 cells for 4 hours before assessing Jurkat cell activation via CD69 expression. Only the SP14-C8 CAR construct induced a significant level of CD69 in the presence of both CHO-EpCAM and MCF7 target cells compared to parental CHO-X cells (FIG. 15E). These observations suggest that both SP14-C8 and SP12-E10 EpCAM CAR-T cells are functionally active and exhibit antigen specific activation and cytotoxicity, and that SP14-C8 is more potent than SP12-E10, consistent with its higher affinity.

SEQUENCES

SEQ ID NO: 1
Human programmed cell depth protein-1, PD-1
MQIPQAPWPVVWAVLQLGRRPGWFAEQKLISEEDLLDSPDRPWNPPTFSP
ALLVVTEGDNATFTCSFSNTSESFVLNWYRMSPSNQTDKLAAFPEDRSQP
GQDCRFRVTQLPCGRDFHMSVVRARRNDCSFSNTSESFVLNWYRMSPSNQ
TDKLAAFPEDRSQPGQDCRFRVTQLPNGRDFHMSVVRARRNDSGTYLCGA
ISLAPKAQIKESLRAELRVTERRAEVPTAHPSPSPRPAGQFQTLVVGVVG
GLLGSLVLLVWVLAVICSRAARGTIGARRTGQPLKEDPSAVPVFSVDYGE
LDFQWREKTPEPPVPCVPEQTEYATIVFPSGMGTSSPARRGSADGPRSAQ
PLRPEDGHCSWPL
SEQ ID NO: 2
N-terminal Myc tag
EQKLISEEDL
SEQ ID NO: 3
scFv protein sequence (X indicates CDRH3 and
CDRL3 sequences)
MWMKIKTGARILALSALTTMMFSASALADIQMTQSPSSLSASVGDRVTIT
CRASQSVSSAVAWYQQKPGKAPKLLIYSASSLYSCVPSRFSGSRSGTDFT
LTISSLQPEDFATYYCXXXXXXXXFGQGTKVGGSSRSSEVQLVESGGGLV
QPGGSLRLSCAASGFNLYYSYIHWVRQAPGKGLEWVASISPSYGYTSYAD
SVKGRFTISADTSKNTAYLQMNSLRAEDTAVYYCARXXXXXXXXXXXXXG
FDYWGQGTLVTVSSTSLEYYPYDVPDYA
SEQ ID NO: 4
Amino acid linker
GGSSRSSEVQLVESGGG
SEQ ID NO: 5
Pembrolizumab from WO2012135408
MGWSCIILFLVATATGVHSELVMTQVQLVQSGVEVKKPGASVKVSCKASG
YTFTNYYMYWVRQAPGQGLEWMGGINPSNGGTNFNEKRKNRVTLTTDSST
TTAYMELKSLQFDDTAVYYCARRDYRFDMGFDYWGQGTTVTVSSASTKGG
GGSGGGGSGGGGSGGGGSGGGGSEIVLTQSPATLSLSPGERATLSCRASK
GVSTSGYSYLHWYQQKPGQAPRLLIYLASYLESGVPARFSGSGSGTDFTL
TISSLEPEDFAVYYCQHSRDLPLTFGGGTKVEIKRLEYYPYDVPDYA
SEQ ID NO: 6
Amino acid linker
GGGGSGGGGSGGGGS
SEQ ID NO: 7
PCR primer
CGATATCAAAGGAGGTACCCAACATG
SEQ ID NO: 8
PCR primer
CGCATAATCCGGCACATCATACGGATAGTAC
SEQUENCE LISTING FREE TEXT
<210> 3
<223> scFv protein sequence (X indicates CDRH3 and
CDRL3 sequences)
<223> Xaa can be any naturally occurring amino
acid
<210> 4
<223> Amino acid linker
<210> 5
<223> Pembrolizumab
<210> 6
<223> Amino acid linker
<210> 7
<223> PCR primer
<210> 8
<223> PCR primer

1. A method of identifying a cell which produces a specific binding partner which binds to a target polypeptide, the method comprising the steps:
(a) expressing a library of binding partners in a population of mammalian cells,
wherein each binding partner comprises a framework and a plurality of variable regions, each plurality of variable regions endowing that binding partner with a specific binding affinity for a target,
wherein each cell in the population of mammalian cells expresses at least one member of the binding partner library from one or more retroviral or lentiviral vectors which have been integrated into the genome of that cell,
wherein each binding partner comprises a first detectable tag,
wherein each binding partner is secreted from the cell in which it is produced, and
wherein each cell in the population of mammalian cells comprises an expression construct comprising a promoter operably linked to a nucleotide sequence encoding the target polypeptide, wherein the target polypeptide optionally comprises a second detectable tag, wherein the expression construct is integrated into the genome of each cell;
(b) optionally removing cells from the population of mammalian cells to which binding partners bind;
(c) expressing or inducing expression of the target polypeptide from the expression construct such that the target polypeptide is displayed on the outer surface of each cell in the population of mammalian cells; and
(d) isolating or identifying cells within the population of mammalian cells to which specific binding partners are bound,
wherein cells to which specific binding partners are bound are isolated or identified using:
(i) flow cytometry, or
(ii) magnetic sorting, or
(iii) a micro-fluidics system wherein cells from the population of mammalian cells are contained within each chamber in a plurality of isolated chambers,
wherein the cells to which specific binding partners are bound are ones which produce specific binding partners which bind to the target polypeptide.

2. The method as claimed in claim 1, wherein in Step (a), the promoter is an inducible promoter and wherein Step (c) comprises:
(c) inducing expression of the target polypeptide from the expression construct such that the target polypeptide is displayed on the outer surface of each cell in the population of mammalian cells.

3. The method as claimed in claim 1, wherein the binding partner is an antibody, antibody fragment, antibody mimetic, an scFv or a soluble T-cell receptor.

4. The method as claimed in claim 1, wherein the method additionally comprises, prior to Step (a), the step of:
contacting the population of mammalian cells with a library of retroviral or lentiviral particles, each particle comprising a retroviral or lentiviral vector encoding a member of a binding partner library and a detectable tag, under conditions such that at least one retroviral or lentiviral vector is integrated into the genome of each cell or substantially each cell in the library.

5. The method as claimed in claim 1, wherein the method additionally comprises, prior to Step (a), the step:
integrating, into the genome of each or substantially each cell in the population of cells, an expression construct comprising a promoter or an inducible promoter operably linked to a nucleotide sequence encoding the target polypeptide and wherein the target polypeptide optionally comprises a second detectable tag.

6. The method as claimed in claim 1, wherein the detectable tag is HA or a Myc tag.

7. The method as claimed in claim 2, wherein the inducible promoter comprises a plurality of Tet operator sequences to which the Tet repressor protein (TetR) is capable of binding.

8. The method as claimed in claim 1, wherein Step (b) and/or Step (c) additionally comprises the step:
contacting all or part of the population of mammalian cells with an excess of capturing cells which express the target polypeptide, and optionally a second detectable tag, but do not express a binding partner,
wherein each capturing cell comprises a label or a fluorescence label which allows the capturing cells to be distinguished from the population of mammalian cells.

9. The method as claimed in claim 1, wherein, in Step (d), specific binding partners are detected using an antibody against the first detectable tag which is labelled with a fluorophore or a paramagnetic particle.

10. The method as claimed in claim 1, wherein each binding partner comprises a first detectable tag, wherein the target polypeptide comprises a second detectable tag, and wherein the first and second detectable tags are detected using independent antibodies to which are attached first and second members of a donor-acceptor FRET pair.

11. The method as claimed in claim 1, wherein, in Step (d), the flow cytometry is FACS or the magnetic sorting is MACS.

12. The method as claimed in claim 1, wherein, in Step (d), the micro-fluidics system is one wherein cells from the population of mammalian cells are contained within each droplet in a plurality of isolated droplets.

13. The method as claimed in claim 12, wherein each droplet comprises 30-50 cells.

14. The method as claimed in claim 12, wherein the droplets have a volume of 1-2000 pL, or 100-1000 pL or about 200 pL.

15. The method as claimed in claim 1, wherein in Step (d), the micro-fluidics system is one wherein cells from the population of mammalian cells are contained within pens which are arranged in an array on a solid substrate or a chip.

16. The method as claimed in claim 15, wherein a micro-fluidics system is used wherein one or more cells from the population of mammalian cells are contained within each pen in an array of isolated pens; wherein non-target polypeptide displaying cells are contacted with or juxtaposed against the edges of the isolated pens in the array such that binding partners which are secreted from the cells within the pens are capable of contacting the non-target polypeptide displaying cells;
and rejecting pens in which cell surface binding of binding partners has been detected on the surface of the non-target polypeptide displaying cells.

17. The method as claimed in claim 1, wherein in Step (d), each chamber comprises one cell which expresses the target polypeptide and a plurality of cells or CHO cells which do not express the target polypeptide; and wherein chambers in which cell surface binding of binding partners has been detected are rejected if cell surface binding of binding partners is detected on more than one cell per chamber.

18. The method as claimed in claim 1, wherein the method additionally comprises the step:
(e) sequencing part of all of the nucleotide sequences in the isolated cells or identified cells which encode the specific binding partners which bind to the target polypeptide.

19. A process for producing a population of mammalian cells, the process comprising the steps:
(A) integrating, into the genome of each or substantially each cell in the population of mammalian cells, an expression construct comprising a promoter or an inducible promoter operably linked to a nucleotide sequence encoding a target polypeptide and wherein the target polypeptide optionally comprises a second detectable tag; and
(B) contacting the population of mammalian cells with a library of retroviral or lentiviral particles, each particle comprising a retroviral or lentiviral vector encoding a member of a binding partner library and a first detectable tag;
so as to produce a population of mammalian cells wherein each cell or substantially each cell in the population of mammalian cells secretes one or more binding partners or antibodies or antibody mimetics, and
wherein each cell in the population of mammalian cells displays or is capable of displaying or is capable of displaying upon induction the target polypeptide, and optionally a second detectable tag, on the outer surface of the cell.
